(19) United States
(12) Patent Application Publication
(10) Pub. No.: US 2026/0315098 A1
(43) Pub. Date: Oct. 15, 2026

(54) GUIDED DEEP-TARGET IN THE BODY LOOPING TOOL-SYSTEM WITHOUT SKIN INCISION

(71) Applicant: Danqing Guo, De Pere, WI (US)

(72) Inventor: Danqing Guo, De Pere, WI (US)

(21) Appl. No.: 19/644,243

(22) Filed: Apr. 10, 2026

Related U.S. Application Data
(60) Provisional application No. 63/787,087, filed on Apr. 11, 2025.

(51) Int. Cl. A61B 17/3203; A61B 17/00; A61B 17/32

(52) U.S. Cl. CPC A61B 17/3203; A61B 17/320016; A61B 2017/00358

(57) ABSTRACT

Apparatus and associated methods relate to surgical device having a conduit complex and a fluid dispenser. In various embodiments, the fluid dispenser includes shafts that may incorporate functionality to control the movement and direction of the conduit complex. The fluid dispenser may incorporate functionality to control the movement and direction of a thread with one magnet end having a locking system. In some embodiments, the conduit complex and the fluid dispenser are operably connected. The conduit complex includes an inner conduit, a middle conduit, and an outer conduit. The inner conduit may, for example, be a metal tube. For example, the inner metal tube with flexible curvature of a distal portion in the body may be configured to be held with the second shaft. The middle conduit with a fixed curvature of a proximal portion outside the body may, for example, have a channel.

### CROSS-REFERENCE TO RELATED APPLICATIONS

This application claims the benefit of U.S. Application Ser. No. 63/787,087, titled “GUIDED DEEP-TARGET IN THE BODY LOOPING TOOL-SYSTEM WITHOUT SKIN INCISION,” filed by Danqing Guo on Apr. 11, 2025.

This application incorporates the entire contents of the foregoing application(s) herein by reference.

### TECHNICAL FIELD

Various embodiments relate generally to surgical devices.

### BACKGROUND

In some industries, precision has been targeted as a desirable feature for a product. For example, various companies in the medical industry design equipment that allow practitioners to use precision to assist their patients.

Precision in diagnostic instruments may, for example, lead to better outcomes for patients. Precise diagnostic instruments may, for example, accurately measure the conditions of a patient, such as temperature and heart rate.

### SUMMARY

Apparatus and associated methods relate to surgical device having a conduit complex and a fluid dispenser. In various embodiments, the fluid dispenser includes shafts that may incorporate functionality to control the movement and direction of the conduit complex. The fluid dispenser may incorporate functionality to control the movement and direction of a thread with one magnet (or iron or solid material) end having a locking system. In some embodiments, the conduit complex and the fluid dispenser are operably connected. The conduit complex includes an inner conduit, a middle conduit, and an outer conduit. The inner conduit may, for example, be a metal tube. For example, the inner metal tube with flexible curvature of a distal portion in the body may be configured to be held with the second shaft. The middle conduit with a fixed curvature of a proximal portion outside the body may, for example, have a channel. The fixed curvature of the proximal portion outside the body may, for example, be configured to be held with the first shaft. The outer conduit may, for example, be a plastic tube. For example, the outer conduit may be configured to cover the channel of the middle conduit. The fluid dispenser includes a first shaft, a second shaft, a third shaft, a one-way valve, a spring, and a rubber plunge connector. The rubber plunge connector may, for example, include a unidirectional valve. For example, the unidirectional valve may be embedded in the rubber plunge connector. A securing system may, for example, be arranged between the first shaft and the second shaft. The second shaft and the third shaft may, for example, form a syringe. For example, the syringe may allow fluid flow from one end of the fluid dispenser to the other end through the one-way valve, the spring, and the rubber plunge connector.

In some embodiments, the second shaft and the third shaft may, for example, form an automatic filling syringe. For example, the automatic filling syringe may allow fluid flow into the body from one end of the fluid dispenser to the other end through the one-way valve and the unidirectional valve in the rubber plunge connector, the central tube in the second shaft, and the inner conduit. Pushing a distal end of the third shaft may, for example, dispense the filled fluid in the third shaft into the body through the unidirectional valve in the rubber plunge connector, the central tube in the second shaft, and connected inner conduit. Simultaneously, the spring may, for example, be compressed. When the pushing of the distal end of the third shaft is stopped, the spring may, for example, extend to push the third shaft back to the original position, creating a negative pressure in the dispenser. The fluid from outside the system may flow, for example, into the dispenser through the one-way valve to refill the dispenser automatically.

The conduits of the conduit complex and the shafts of the fluid dispenser may, for example, be operably connected. For example, the first and second shafts control the relative movements of the conduits. The first and second shafts may, for example, cooperatively control the relative movements of the conduits. This may, for example, advantageously guide the direction of the tip of the conduit complex. The third shaft may, for example, operatively function as a fluid-dispensation syringe. For example, the third shaft may function as hydrodissection to open the space between the anatomy in a patient to let the conduit complex moving towards a target area under ultraminimally invasive conditions.

In some examples, the first and second shafts may be operably coupled to one of the conduits. Each of the first and second shafts may, for example, be configured to rotate the conduit it is coupled with 360° while the conduit is inside the body. For example, the 360° rotation may advantageously provide control of the direction of the tip of the conduit complex at the practitioner's discretion. In some embodiments, a lock device may be arranged between the first shaft and the second shaft. This lock device may, for example, immobilize the first shaft and the second shaft to avoid relative movement after desired direction of the tip is acquired.

In an illustrative example, the conduit complex and the fluid dispenser may operably loop a thread around a target area inside a patient. The shafts may, for example, control the thread releasably inserted into the patient via the conduits. The target area may, for example, be a fascia, scarring tissue, ligament, etc., compressing on a nerve, vessels such as arteries or veins that need to be released, or bones that need to be cut. For example, the movement of the conduit complex may be guided by ultrasound, CT, or other guidance means. In some embodiments, the shafts may advantageously rotate, extend, and retract the conduits while they are in a patient. This may, for example, advantageously provide the practitioner significant movement capabilities with the device.

In some examples, the conduit complex may be inserted near the target area to deposit a thread. The thread may, for example, be coupled to a magnet at one end. For example, the magnet and the thread are positioned around one side of the target area. In some implementations, the conduit complex may receive a magnetic bar that extends through the complex. The magnetic bar may, for example, include a locking system to secure the thread. The magnetic bar may, for example, be positioned on the other side of the target area to couple with the magnet at the end of the thread. The coupling of the magnetic bar and the magnet may, for example, form a loop around the target area. The loop may, for example, be secured by movement of the magnetic bar and the conduit outside of the magnetic bar forward to secure the magnet end of the thread inside the conduit.

Various embodiments may achieve one or more advantages. For example, the conduit complex and fluid dispenser advantageously provide a practitioner access deep within a patient's body without having to cause significant trauma to the patient. For example, instead of needing surgical incision and multiple cuts to perform an operation to treat a target area (e.g., release the compression, break hard tissue, etc.), the conduit complex may be inserted into the patient through a single, needle-sized skin poke to form the loop around the target area to treat the target area.

In various embodiments, selectively operating the target area while avoiding unnecessary iatrogenic damage may, for example, advantageously and significantly limit the amount of trauma endured by a patient. In some implementations, the surgical device may advantageously reduce the cost of treating a patient due to ultraminimally invasive surgeries not requiring an operating room, monitoring system, and/or an anesthesiologist. In various examples, the surgical device may advantageously reduce the number of staff required to perform a procedure. In some examples, the surgical device may advantageously reduce the healing time needed for the patient. For example, there is limited, if any, skin-needle-poking mark. In some embodiments, there may advantageously be no surgical incision-like scarring.

In various implementations, the surgical device may be used in numerous operations. For example, the conduit complex, in combination with ultrasound, CT, or other guidance means, advantageously enables targeting areas deep within the body. This may, for example, advantageously enable the surgical device to be used in any operation where releasing pressure on a nerve, releasing a certain target deep inside the body, or sawing break and hard tissues such as a bone deep inside the body without incising skin is necessary.

In various embodiments, the surgical device may be configured for use in procedures performed in musculoskeletal and/or subcutaneous tissues, including (by way of example and not limitation) fascia, ligament, scar tissue, peri-neural planes, and other soft tissues adjacent to nerves, vessels, tendons, or bone. The conduit complex may be advanced through tissue planes under imaging guidance and, in some implementations, may employ hydrodissection to create or expand a working space within soft tissue to facilitate controlled positioning of a thread and/or other treatment element at or around a target structure. In various examples, the conduit complex is configured to travel through and operate within solid tissue and tissue planes, with the target looping and/or cutting action occurring at a target located in musculoskeletal or subcutaneous anatomy.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary surgical device employed in an illustrative use-case scenario.

FIG. 2 depicts an illustrative surgical device.

FIG. 3 depicts an illustrative exploded view and illustrative assembled view of a fluid dispenser of a surgical device.

FIG. 4 depicts an illustrative conduit complex of a surgical device.

FIG. 5 depicts an illustrative cross-sectional view and an illustrative long-axis view of a conduit complex of a surgical device.

FIG. 6 depicts an illustrative view of a fluid dispenser of a surgical device.

FIG. 7 depicts illustrative views of a thread moved out of a conduit complex of a surgical device.

FIG. 8 depicts illustrative views of a thread moving into a conduit complex of a surgical device.

FIG. 9 depicts illustrative views of a locking system of a magnet end of thread and magnetic bar of a surgical device.

FIG. 10 depicts illustrative views of a locking end of a magnetic bar of a surgical device.

FIG. 11 is a flowchart illustrating an illustrative method of using a surgical device.

FIG. 12 depicts an exemplary surgical device employed in an illustrative use-case scenario.

FIG. 13 depicts an exemplary surgical device employed in an illustrative use-case scenario.

FIG. 14 depicts an exemplary surgical device employed in an illustrative use-case scenario.

FIG. 15 depicts an exemplary surgical device employed in an illustrative use-case scenario.

FIG. 16 depicts an exemplary surgical device employed in an illustrative use-case scenario.

FIG. 17 depicts an exemplary surgical device employed in an illustrative use-case scenario.

FIG. 18 depicts an exemplary surgical device employed in an illustrative use-case scenario.

FIG. 19 depicts an exemplary surgical device employed in an illustrative use-case scenario.

FIG. 20 depicts an exemplary surgical device employed in an illustrative use-case scenario.

FIG. 21 depicts an exemplary surgical device employed in an illustrative use-case scenario.

FIG. 22 depicts an exemplary locking system of a surgical device.

FIG. 23 depicts an exemplary tube of a surgical device.

FIG. 24 depicts an exemplary surgical device employed in an illustrative use-case scenario.

FIG. 25 depicts an exemplary surgical device.

FIG. 26 depicts an illustrative hydrodissection tube with an end-hook and end-knob thread looping system.

FIG. 27 depicts an illustrative surgical device for picking up an end of a thread within a body to form target looping.

FIG. 28 depicts an exemplary surgical device employed in an illustrative use-case scenario.

FIG. 29 depicts an exemplary surgical device employed in an illustrative use-case scenario.

FIG. 30 depicts an exemplary surgical device employed in an illustrative use-case scenario.

FIG. 31 depicts an exemplary surgical device employed in an illustrative use-case scenario.

FIG. 32A depicts an exemplary surgical device employed in an illustrative use-case scenario.

FIG. 32B depicts an exemplary surgical device employed in an illustrative use-case scenario.

FIG. 33 depicts an exploded view of an illustrative open/close controllable loop surgical device with a hydrodissection tunnel.

FIG. 34 depicts an assembled view of an illustrative open/close controllable loop surgical device with a hydrodissection tunnel.

FIG. 35 depicts an exemplary surgical device employed in an illustrative use-case scenario.

FIG. 36 depicts an exemplary surgical device employed in an illustrative use-case scenario.

FIG. 37 depicts an exemplary surgical device employed in an illustrative use-case scenario.

FIG. 38 depicts an exemplary surgical device employed in an illustrative use-case scenario.

FIG. 39 depicts an exemplary surgical device employed in an illustrative use-case scenario.

FIG. 40 depicts an exemplary surgical device employed in an illustrative use-case scenario.

FIG. 41 depicts an exemplary attachment system of an illustrative surgical device.

FIG. 42 depicts an exemplary attachment system of an illustrative surgical device.

Like reference symbols in the various drawings indicate like elements.

### DETAILED DESCRIPTION OF ILLUSTRATIVE EMBODIMENTS

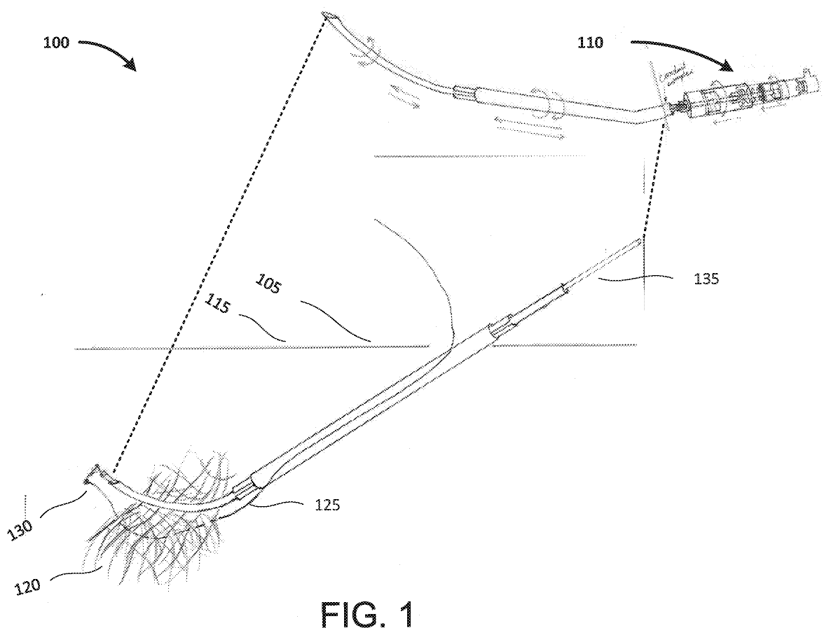

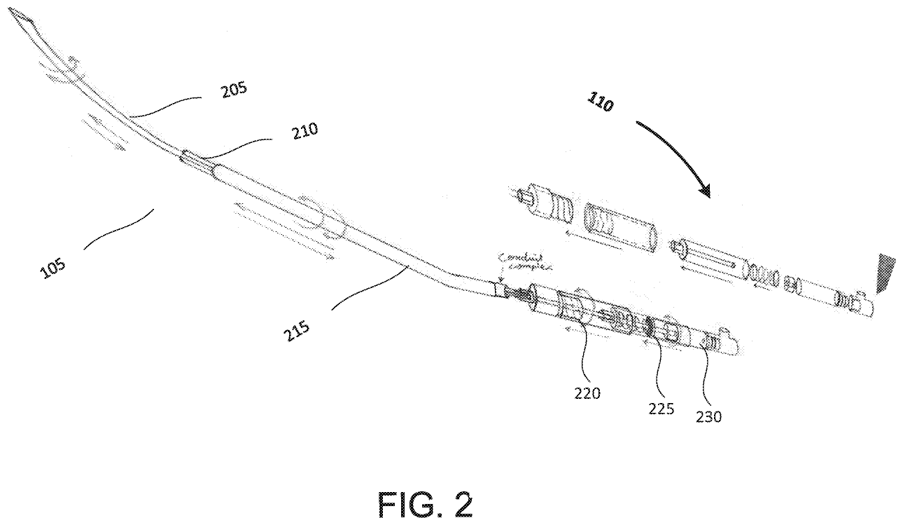

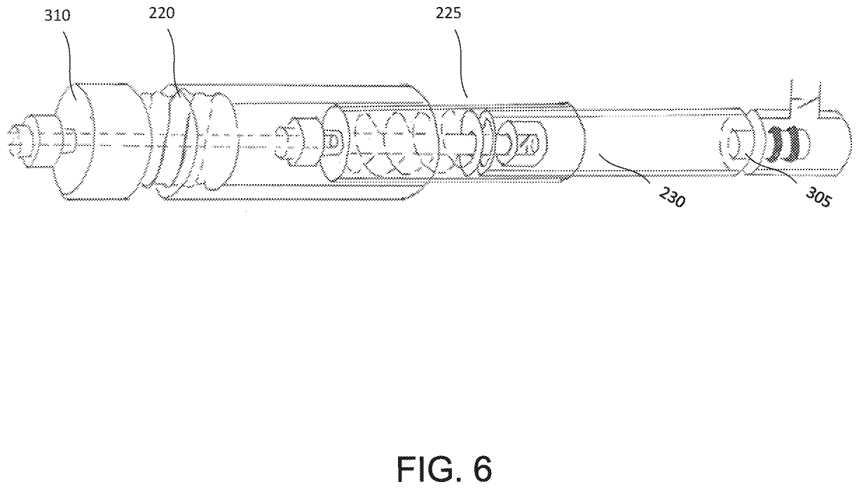

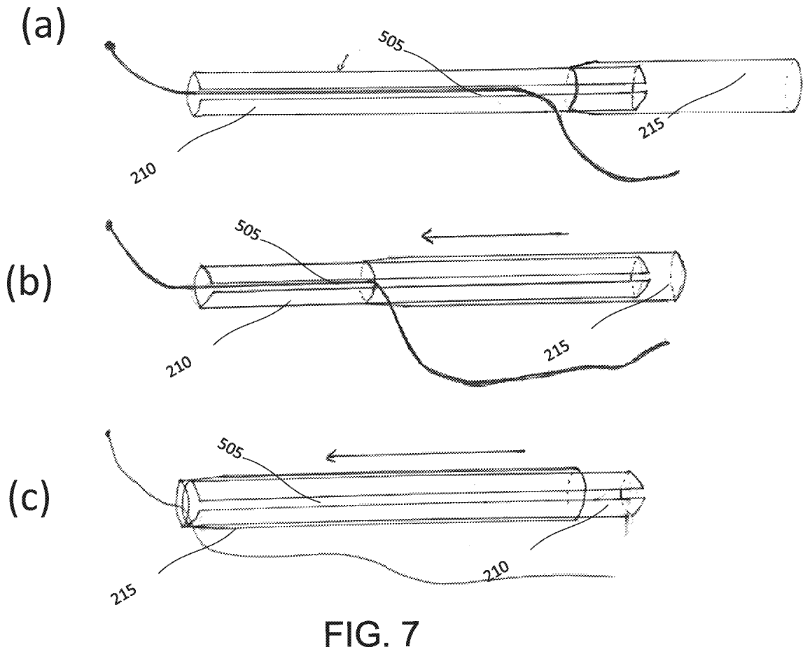

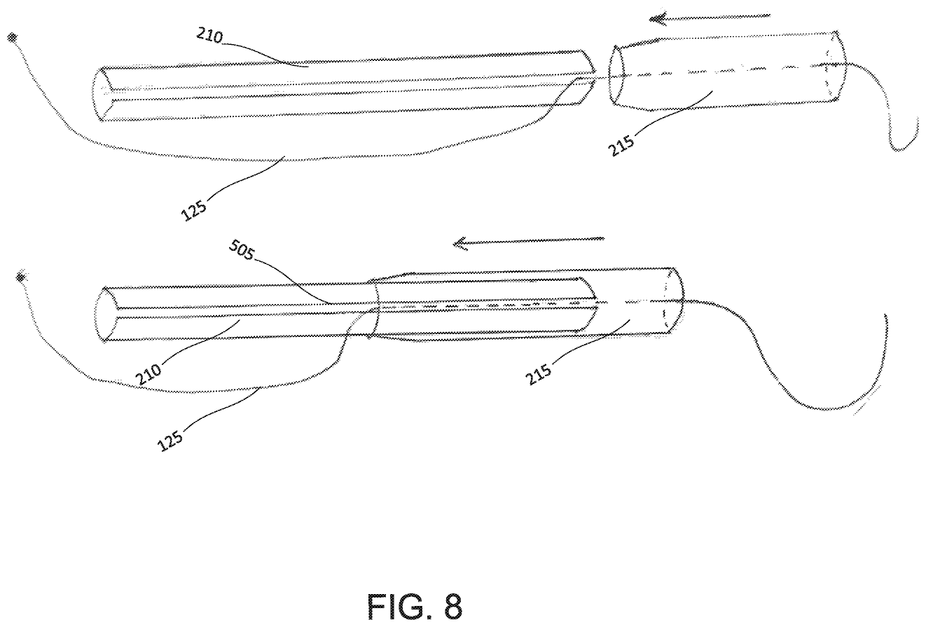

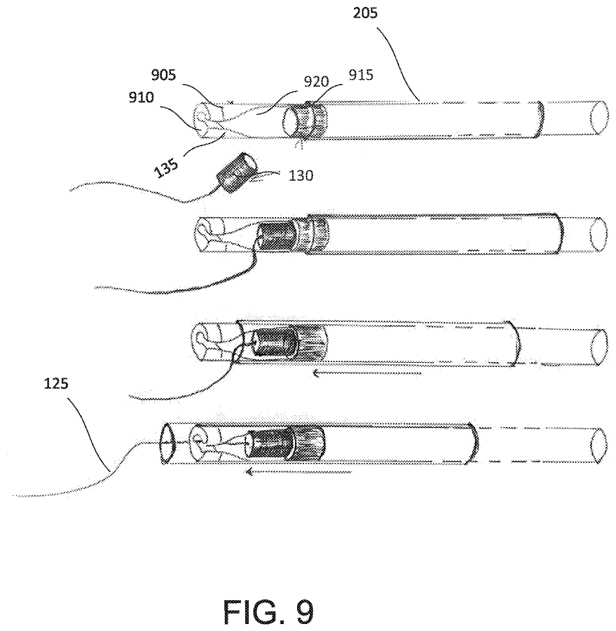

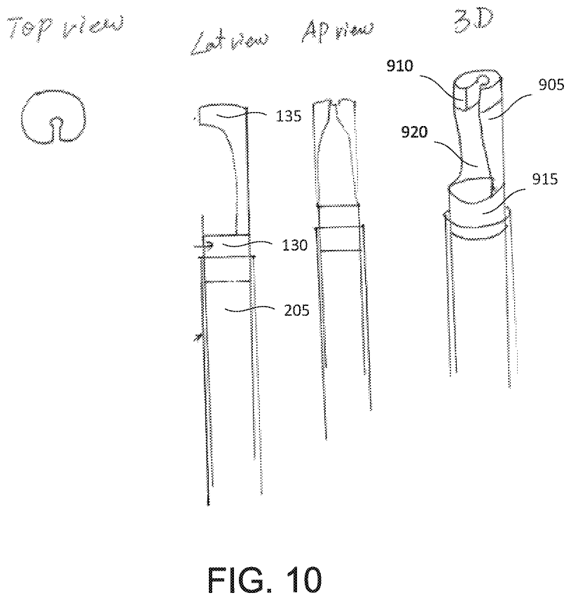

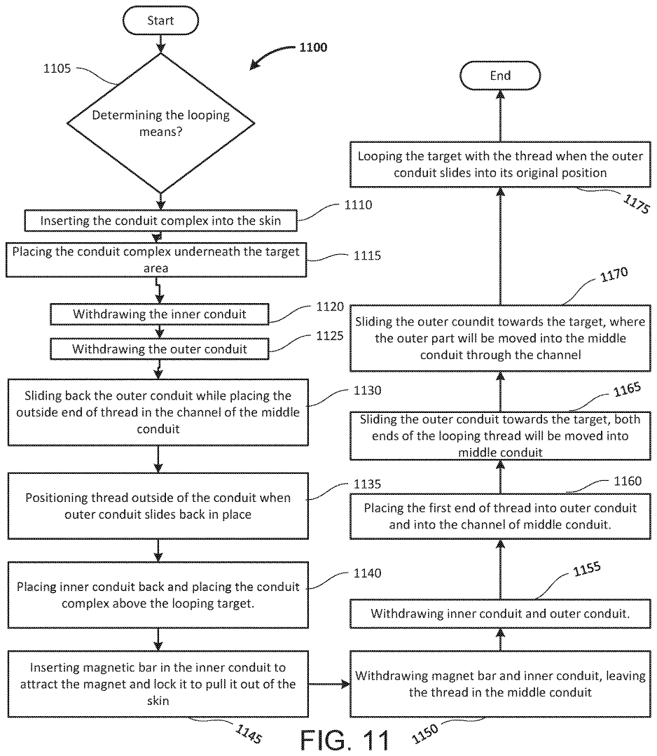

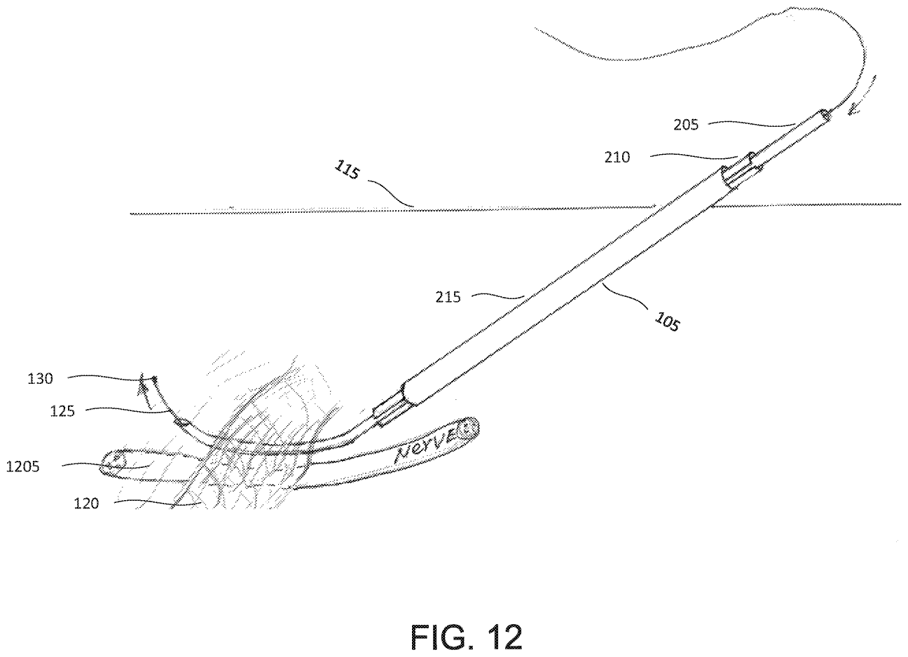

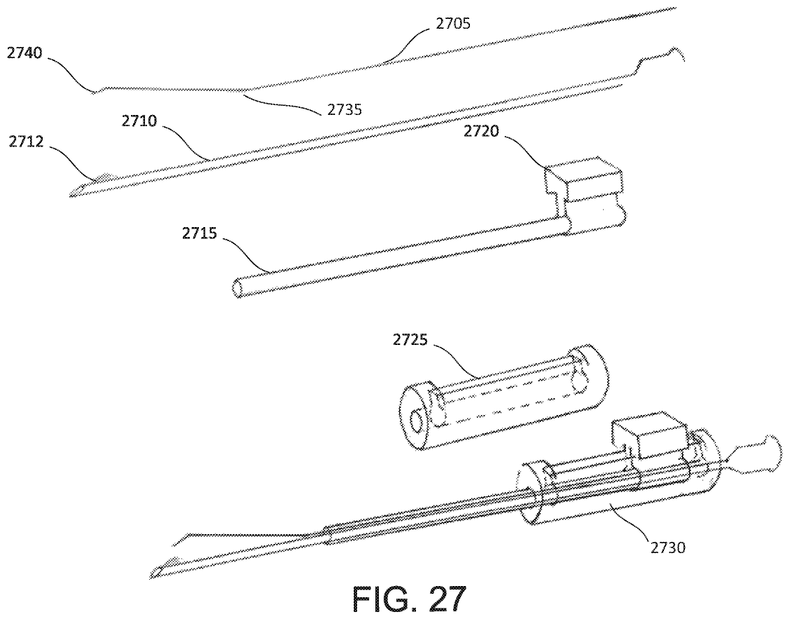

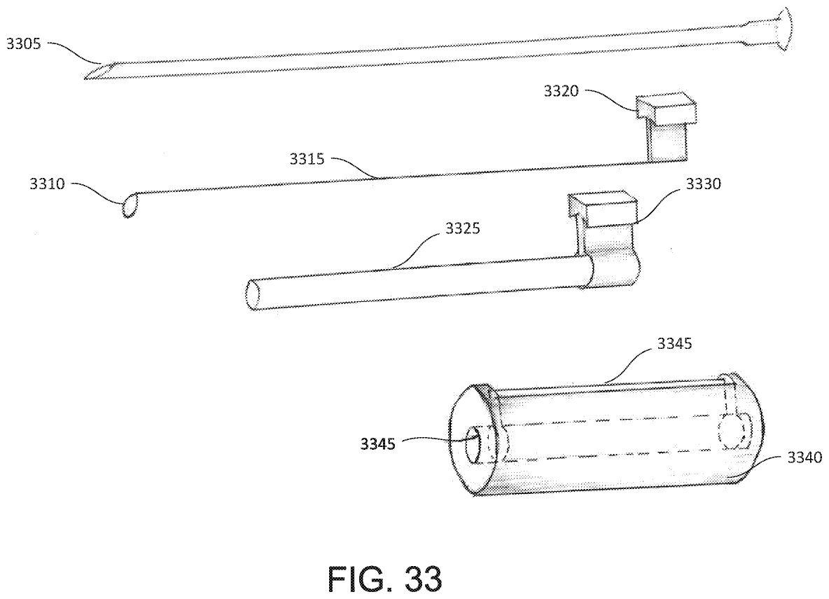

To aid understanding, this document is organized as follows. First, to help introduce discussion of various embodiments, an exemplary surgical device employed in an illustrative use-case scenario is introduced with reference to FIG. 1. Second, that introduction leads into a description with reference to FIGS. 2-6 of some exemplary embodiments of a fluid dispenser of a surgical device and a conduit complex of a surgical device. Third, with reference to FIGS. 7-8, discussion turns to exemplary embodiments that illustrate a thread moving through conduits of a conduit complex. Fourth, with reference to FIGS. 9 and 10, the discussion turns to exemplary embodiments that illustrate a magnetic bar and a magnet coupled to a thread. Fifth, and with reference to FIG. 11, this document describes an exemplary method for forming a loop around a target area with the surgical device. Sixth, the document discusses further embodiments, exemplary applications and aspects relating to a surgical device in FIGS. 12-26. Seventh, the document further discusses embodiments directed to an illustrative surgical device in FIGS. 27-32. Eighth, the document also discusses embodiments directed to an illustrative surgical device in FIGS. 33-40.

FIG. 1 depicts an exemplary surgical device employed in an illustrative use-case scenario. A surgical device 100 includes a conduit complex 105 and a fluid dispenser 110. The conduit complex 105 and the fluid dispenser 110 are operably, fluidly, and detachably connected. The fluid dispenser 110 may, for example, be configured to control the conduit complex 105 while the conduit complex 105 is inserted into a patient's body through skin 115. For example, the dispenser 110 may be configured to direct the conduit complex 105 to a target area 120.

In some implementations, the fluid dispenser 110 may be detached from the conduit complex 105. The conduit complex 105 may, for example, be configured to receive a thread 125 with a magnet 130 at one end of the thread 125. The conduit complex 105 may, for example, controllably position the thread 125 under the target area 120. For example, the thread 125 may be positioned under a fascia and/or ligament compressing on a nerve. In some examples, the conduit complex 105 may be configured to position a magnetic bar 135. The magnetic bar 135 may, for example, be positioned by the conduit complex 105 to couple with the magnet 130 and locking system, then withdrawing the magnetic bar 135 with the locked magnet 130 end of the thread 125 to move the magnet 130 end of the thread 125 outside the skin 115, forming a loop around the target area 120 with the thread 125. In various implementations, the loop formed by the thread 125 may, for example, advantageously enable the target area 120 to be manipulated. For example, the target area 120 may be cut by the thread 125.

In some embodiments, the surgical device includes the conduit complex 105 operably connected to the fluid dispenser 110 to control the thread 125 releasably inserted into the skin 115 of a patient to the target area 120 in the deep body.

In various embodiments, the device is directed to a soft tissue surgical system including a needle having an outer diameter; a lumen extending through the needle; an adjustable loop mechanism positioned at a distal end of the needle; a flexible wire configured to be advanced through the adjustable loop mechanism; wherein the adjustable loop mechanism includes at least two controller components disposed within the lumen and configured to move telescopically relative to one another to transition the loop mechanism between a closed configuration for insertion and an open configuration after insertion into a body; wherein movement of the flexible wire relative to the adjustable loop mechanism is configured to tighten the loop mechanism and close around the flexible wire; and wherein the at least two controller components are operably controlled by two controllers.

FIG. 2 depicts an illustrative surgical device. The conduit complex 105 and the fluid dispenser 110 are operably connected. The conduit complex 105 includes an inner conduit 205, middle conduit 210, and outer conduit 215. The inner conduit 205 may, for example, include an adjustable, reversible curvature at a distal portion of the inner conduit 205. The curvature may, for example, be forcibly shaped straight when the curvature is in the middle conduit 210. The degree of the curvature of the distal portion may, for example, be controlled by the position of the inner conduit 205, the middle conduit 210, and the outer conduit 215. For example, the curvature may be controlled through the extending and retracting of the inner conduit 205 in relation to the middle conduit 210 and the outer conduit 215.

The fluid dispenser 110 includes a first shaft 220, second shaft 225, and third shaft 230. The second shaft 225 may, for example, operably control the inner conduit 205. For example, rotating the second shaft 225 may rotate the inner conduit 205 to change the direction of the tip of the inner conduit 205. In some implementations, extending the second shaft 225 may extend the inner conduit 205. Retracting the second shaft 225 may, for example, retract the inner conduit 205. The degrees of movements may, for example, advantageously provide greater control and flexibility of the inner conduit 205 while the inner conduit 205 is within the patient.

In some embodiments, the first shaft 220 may operably control middle conduit 210 and the outer conduit 215 simultaneously. For example, rotating the first shaft 220 may rotate the middle conduit 210 and the outer conduit 215 simultaneously. In some implementations, extending or retracting the first shaft 220 may extend or retract the middle conduit 210 and the outer conduit 215, which may reduce or increase the curvature and length of the inner conduit 205. Retracting the first shaft 220 may, for example, retract the middle conduit 210 and the outer conduit 215 simultaneously. For example, the retraction of the first shaft 220 may advantageously increase the curvature of the inner conduit 205 and increase the length of the conduit complex 105.

In some embodiments, the middle conduit 210 may, for example, include a rigid curvature at a proximal portion of middle conduit 210 that may create movement of the conduit complex 105 in the body without adjusting dispenser position outside body. The rigid curvature may, for example, create an angle between the dispenser and the skin surface. The angle may, for expel, make manipulation of the dispenser easier. Simultaneously, the angle may, for example, enable horizontal positions of the conduit complex 105 in the body when needed. The middle conduit 210 may, for example, connect to the first shaft 220 through a connector. The first shaft 220 may, for example, rotate. When the first shaft 220 rotates, the first shaft 220 may change from one plane to two different planes between the dispenser plane and the conduit system plane. This may, for example, advantageously make the device easier to manipulate. For example, in situations when there is no longer enough space to manipulate the fluid dispenser 110 and the conduit complex 105, it may be beneficial to change the fluid dispenser 110 plane to make an angle with the conduit complex 105.

In various embodiments, the third shaft 230 may operably control fluid dispensation through two one-way adapters. One of the one-way adapters may, for example, be a rubber plunge level The other one-way adapter may, for example, be located at the inlet of the fluid. In some implementations, the two one-way adapters may, for example, be configured to operate as a syringe. A spring may, for example, be configured to automatically refill the third shaft 230.

In some embodiments, the first shaft 220 may operably control the middle conduit 210 and the outer conduit 215 simultaneously. This arrangement may, for example, advantageously provide increased movement capabilities of the device 100. For example, the first shaft 220 may extend and retract the middle conduit 210 and the outer conduit 215 simultaneously in the patient. The first shaft 220 may, for example, rotate the middle conduit 210 and the outer conduit 215 360° simultaneously in the patient. The extension, retraction, and rotation of the middle conduit 210 and the outer conduit 215 simultaneously advantageously provides different directional capabilities as needed to reach the target area 120. The second shaft 225 may, for example, extend and retract the inner conduit 205. This may, for example, advantageously alter the curvature of the inner conduit 205. For example, the second shaft 225 may extend and retract the inner conduit 205 in the patient. The second shaft 225 may, for example, rotate the inner conduit 205 360° in the patient. The extension, retraction, rotation, and altering of the curvature of the inner conduit 205 advantageously provides different directional capabilities to reach the target area 120. The degrees of movement of the inner conduit 205, the middle conduit 210, and the outer conduit 215 may, for example, advantageously allow a practitioner to precisely reach the target area 120 in a patient. This may, for example, advantageously allow a practitioner to reach the target area 120 quickly without having to reinsert the device 100 for a new position.

In various implementations, the first shaft 220 may be configured to control the middle conduit 210 and the outer conduit 215 simultaneously. In some examples, the second shaft 225 may be configured to control the inner conduit 205. The third shaft 230 may, for example, be configured to function as a plunger of the fluid dispenser 110.

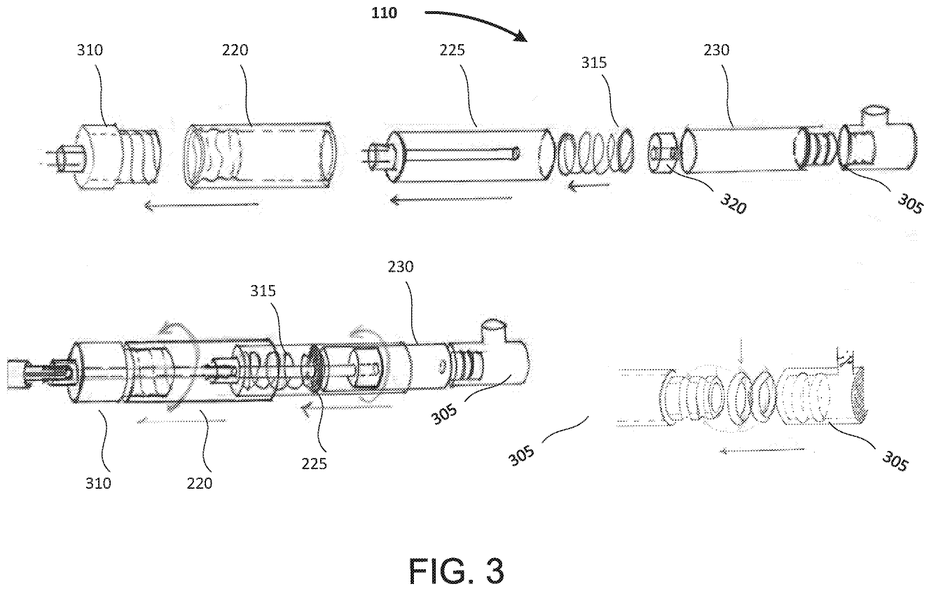

FIG. 3 depicts an illustrative exploded view and illustrative assembled view of a fluid dispenser of a surgical device. The fluid dispenser 110 includes the first shaft 220, the second shaft 225, the third shaft 230, one-way adapter 305, a connector 310, a spring 315, and a rubber stopper one-way adapter 320. In some embodiments, the first shaft 220, the second shaft 225, the third shaft 230, the one-way adapter 305, the connector 310, the spring 315, and the rubber stopper one-way adapter 320 may be fluidly connected. The first shaft 220, the second shaft 25, the third shaft 230, the one-way adapter 305, the spring 315, and the rubber stopper one-way adapter 320 may, for example, be operably connected.

The one-way adapter 305 may, for example, be configured to transport fluid in one direction into the third shaft 230. The rubber stopper one-way adapter 320 may, for example, be configured to transport fluid in one direction into the body through the conduit complex 105 for hydrodissection. This may, for example, advantageously prevent backflow of fluid through the fluid dispenser 110. For example, preventing backflow ensures the accuracy of the amount of fluid in the fluid dispenser 110 as fluid loss does not occur.

In some embodiments, the spring 315 is arranged between the second shaft 225 and the third shaft 230. The spring 315 may, for example, be operably connected to proximal end of the third shaft 230. For example, the spring 315 may be operably configured to return the third shaft 230 back to its original position to refill the third shaft 230 automatically after the third shaft 230 is pushed to dispense fluid into the body of the patient. In some implementations, the engagement of the one-way adapter 305 and the rubber stopper one-way adapter 320 with the third shaft 230 may, for example, be configured to function as an automatic refill one-way dispensing syringe. When pushing the one-way adapter 305 proximally, the fluid may be dispensed into the body through the inner conduit 205. When releasing the one-way adapter 305, the spring 315 may, for example, return the third shaft 230 to its original position. For example, this return of the third shaft 230 may create a negative pressure in the third shaft 230. The negative pressure may, for example, advantageously suction fluid into the third shaft 230 through the one-way adapter 305. The suctioned fluid may, for example, be stored in the third shaft 230 for later use. The fluid may, for example, flow in the third shaft 230 and the inner conduit 205. The first shaft 220, the second shaft 225, the middle conduit 210, and the outer conduit 215 may, for example, be configured to control the tip of the conduit complex 105 and the moment of the thread 125 position.

The fluid may, for example, flow through the tube to the inner conduit 205, which is engaged with the second shaft 225. In various embodiments, when the one-way adapter is no longer pushed, the spring 315 may return the third shaft 230 back to its original position. In some examples, when the spring 315 returns the third shaft 230 back to its original position, the third shaft 230 may advantageously be filled through the one-way adapter automatically. This may, for example, advantageously prepare the next dispensing of fluid.

In some implementations, the connector 310 couples with the middle conduit 210. The inner conduit 205 may, for example, extend through the connector 310 to couple with the second shaft 225. The connector 310 may, for example, fluidly couple the conduit complex 105 and the fluid dispenser 110. For example, fluid may travel through the one-way adapter 305, the rubber stopper one-way adapter 320, the tube in the second shaft 225, the inner conduit 205, and the connector 310 to the conduit complex 105. The connector 310 may, for example, couple with the first shaft 220 through a fastener connection. In some examples, the first shaft 220 may detach from the connector 310 and shift towards the second shaft 225 to expose the engagement of the inner conduit 205 to the second shaft 225. This may, for example, advantageously enable detachment from the inner conduit from the second shaft In some embodiments, the one-way adapter 305 may, for example, connect to the third shaft 230 with two rubber bends sealed at connection. The one-way adapter 305 may, for example, have a 360-degree rotation that may, for example, help the practitioner adjust the inlet tubing position as needed.

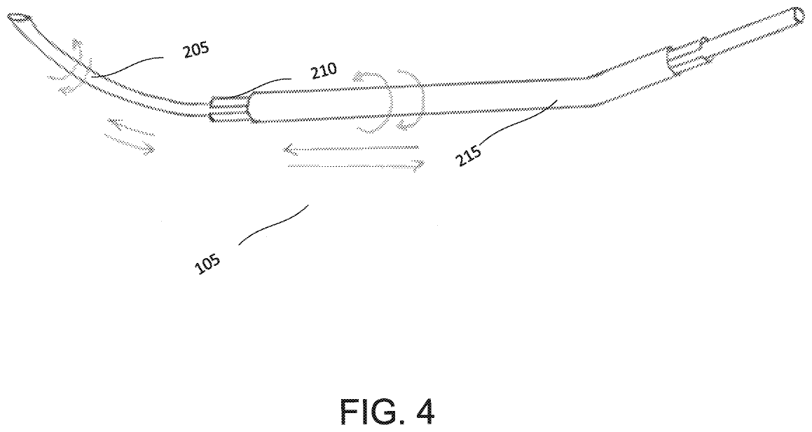

FIG. 4 depicts an illustrative conduit complex of a surgical device. The conduit complex 105 includes the inner conduit 205, the middle conduit 210, and the outer conduit 215. The inner conduit 205, the middle conduit 210, and the outer conduit 215 may, for example, be configured to rotate, extend and/or retract. For example, the rotation, extension, retraction, and/or altering of the curvature of the inner conduit 205, the middle conduit 210, and/or the outer conduit 215 may provide precise movement patterns that advantageously assists in looping the target area 120. In some implementations, the movements of the inner conduit 205 may be cooperated with the movements of the middle conduit 210 and the outer conduit 215. By moving the position of the inner conduit 205 or by cooperating the movements of the inner conduit 205 with the movements of the middle conduit 210 and the outer conduit 215 via rotation, extension and retraction, the thread 125 may be looped around the target area 120.

In some implementations, the cooperation of the inner conduit 205, the middle conduit 210, and the outer conduit 215 may provide precise movement patterns that advantageously assists in looping the target area 120.

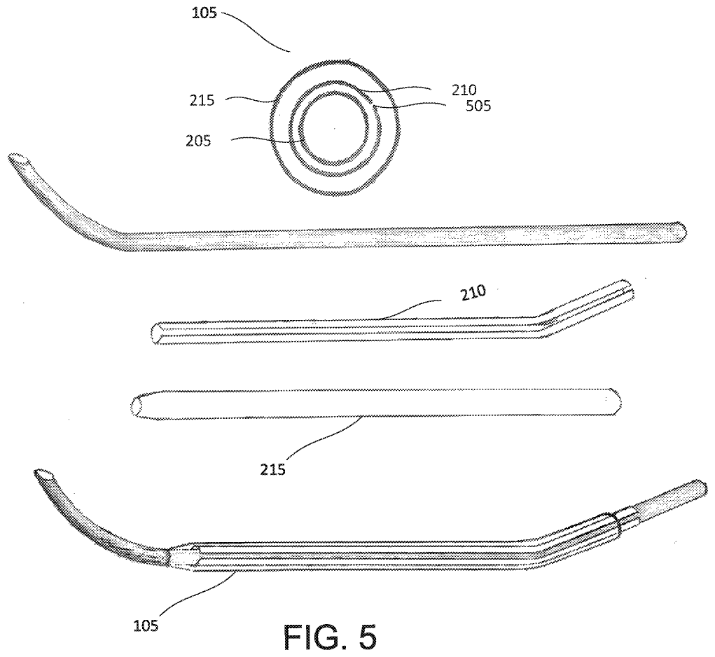

FIG. 5 depicts an illustrative cross-sectional view and an illustrative long-axis view of a conduit complex of a surgical device. The inner conduit 205 may, for example, include a memorable and adjustable curvature at a distal portion of the inner conduit 205. The inner conduit 205, may, in some examples, be forcibly re-shaped straight when in the middle conduit 210. The degree of the curvature of the distal portion may, for example, be controlled by the position of the inner conduit 205, the middle conduit 210, and the outer conduit 215. For example, the curvature may be controlled through the extending and retracting of the inner conduit 205 in relation to the middle conduit 210 and the outer conduit 215.

In some embodiments, the middle conduit 210 defines a channel 505. The channel 505 may, for example, be configured to receive the thread 125. In some examples, the thread 125 may be released from the middle conduit 210 or be moved into the middle conduit 210 via the outer conduit 215 to avoid withdrawing the conduit complex 105 completely and redundantly. In some embodiments, the channel 505 may retain the thread in the conduit complex 105. The looping of the target area 120 may, for example, be exposed outside the conduit complex 105. In some embodiments, if the target needs to be incised, the movement of the thread 125 may occur in the middle conduit 210. This may, for example, significantly decrease the iatrogenic trauma.

In various embodiments, the middle conduit 210 may, for example, include irreversible curvature at a proximal portion of middle conduit 210 that may create movement of the conduit complex 105. This curvature may, for example, create an angle between the dispenser and skin surface that may make manipulation of dispenser easily. Simultaneously, the angle may, in some examples, keep the conduit complex 105 horizontal when needed. The middle conduit 210 may, for example, connect to the first shaft 220 through a connector. The first shaft 220 may, for example, rotate. When the first shaft 220 rotates, the first shaft 220 may change from one plane to two different planes between the dispenser plane and the conduit complex plane. This may, for example, advantageously make the device easier to manipulate. For example, in situations when there is no longer enough space to manipulate the fluid dispenser 110 and the conduit complex 105, it may be beneficial to change the fluid dispenser 110 plane to make an angle with the conduit complex 105.

In some embodiments, the outer conduit 215 may be flexibly bendable. The outer conduit 215 may, for example, be made of plastic. For example, the outer conduit 215 may include a smooth wedge-shape distal end covers the distal end of middle conduit 210 and tightly seamlessly connects to inner conduit 205 that may avoid iatrogenic trauma to surrounding tissue when conduit complex moves in the body.

FIG. 7 depicts illustrative views of a thread moved out of a conduit complex of a surgical device. The conduit complex 105 is configured to receive the thread 125. The conduit complex 105 may, for example, position the thread 125 in the patient. The magnet 130 of the thread 125 may, for example, be placed at the distal end of the target area 120. The inner conduit 205 may, for example, be withdrawn while keeping the magnet 130 in place at the distal end of the target area 120. In some examples, the middle conduit 210, the outer conduit 215, and the thread 125 in the middle conduit 210 may remain in place. This positioning may, for example, be a result of needing to use the conduit complex 105 to finish looping the upper part of the target area 120 through the same conduit to avoid further penetrating skin or course through body tissue. In some examples, the thread 125 may be temporarily removed from the conduit complex 105 to free the conduit complex 105.

As depicted in (a) in FIG. 7, the inner conduit 205 may be removed from the conduit complex 105. The thread 125 may, for example, be inserted into the patient by moving the outer conduit 215 towards the target area 120. For example, the movement of the outer conduit 215 may engage the outer conduit 215 with the thread 125 extending from the channel 505. As depicted in (b) and (c) in FIG. 7, the outer conduit 215 may place the thread 125 out of the conduit complex 105. This placement of the thread 125 may, for example, advantageously position the thread 125 in a desirable location and free the conduit complex 105 to continue to perform the procedure on the patient and avoid replacing the conduit complex 105, minimizing the surgical trauma.

FIG. 8 depicts illustrative views of a thread moving into a conduit complex of a surgical device. The middle conduit 210 and the outer conduit 215 may, for example, be configured to return the thread 125. For example, the thread 125 may extend through the outer conduit 215. As depicted in (d) and (e) in FIG. 8, the thread 125 may be received by the channel 505 of the middle conduit 210 as the outer conduit 215 is moved in the direction of the middle conduit 210 towards the target.

FIG. 9 depicts illustrative views of a locking system of a magnet end of thread and magnetic bar of a surgical device. FIG. 10 depicts illustrative views of a locking end of a magnetic bar of a surgical device. A locking mechanism, as depicted in FIG. 9, may be configured to prevent detachment of the magnetic force between the magnet 130 end of the thread 125 and the magnetic bar 135. The magnetic bar 135 may, for example, be a bar. For example, the bar may be metal or plastic. In some examples, the bar may be telescoped with a tube. The tube may, for example, be metal or plastic. The end of the magnetic bar 135 may, for example, be configured to couple with the magnet 130 at the end of the thread 125. In some examples, this may, stop magnet 130 from passing through. This may, for example, only enable the thread to pass through the hole in the center of the end of the bar.

As depicted in FIGS. 9 and 10, the magnetic bar 135 includes a coupling portion 905. The coupling portion 905 may include a top portion 910 and a bottom portion 915. The coupling portion 905 may define an opening 920. The top portion 910 may, for example, be solid with a center hole. The bottom portion 915 may, for example, be configured to couple with the magnet 130 on the thread 125. The outside tube of the magnetic bar 135 may, for example, slide towards the end of the magnetic bar 135 as depicted in FIG. 9. In some examples, the thread 125 may be positioned into the channel 505 and into the center hole of the top portion 910. The thread 125 may, for example, be secured into the magnetic bar 135.

In some examples, the thread may be pushed into the opening 920. The thread may, for example, continue to slide the tube towards the end of the magnetic bar 135. In some embodiments, the tube may be the inner conduit 205. In various embodiments, the tube may not be the inner conduit 205.

In some embodiments, the magnet 130 is coupled to the end of the thread 125. The magnetic bar 135 may, for example, be configured to be insertable through the inner conduit 205. For example, the magnet 130 may be placed in a location near the distal end of the target area 120 after the conduit complex 105 reaches the distal end of the target area 120 superficial to the target area 120. In some embodiments, the magnet 130 may be replaced with iron.

In some examples, the magnetic bar 135 may be inserted into the inner conduit 205 to couple with the magnet 130. The coupling of the magnet 130 and the magnetic bar 135 may, for example, advantageously form the loop around the target area 120. This loop may, for example, beneficially provide a means to cut the target area 120 with the thread 125.

In some implementations, the thread 125 may include a circular portion at one end. The circular portion may, for example, be configured to receive a hook. In some examples, the hook may be coupled to the end of a bar. The bar may, for example, be configured to be insertable through the inner conduit 205. In some implementations, the bar may couple the hook to the circular portion at the end of the thread 125. This coupling may, for example, advantageously form the loop around the target area 120. For example, the coupling of the circular portion and the hook may be advantageous in patients with components in their body that may be magnetic. The hook and loop coupling may, for example, beneficially provide another type of connection to form the loops when magnets are not readily available.

FIG. 11 is a flowchart illustrating an illustrative method of using a surgical device. The method 1100 starts with determining the looping means in step 1105. In some implementations, the magnet 130 and the magnetic bar 135 may be used to loop the target area 120. In some examples, the circular portion at one end of the thread 125 and the hook coupled to the bar may be used to loop the target area 120. The conduit complex is inserted into the skin in step 1110. The conduit complex is placed underneath the target area and reaches a distal pointe of the target area through the inner conduit in step 1115. The inner conduit is then withdrawn in step 1120, moving the thread to the middle conduit. The outer conduit is withdrawn in step 1125. The outer conduit is slid back while placing the outside end of a thread in a channel of the middle conduit in step 1130. The thread is positioned outside of the conduits when the outer conduit is slid back in place in step 1135. The inner conduit is positioned back in place and the conduit complex is placed above the looping target and advanced to the distal point of the target in step 1140. The magnetic bar is inserted inside the inner conduit to attract the magnet on the thread and then slide the tube conduit on the magnetic bar to lock the thread, then pull the magnetic bar and inside end of the thread out of the skin in step 1145. The magnetic bar is withdrawn from the inner conduit, leaving the thread in the middle conduit in step 1150. The inner conduit and the outer conduit is withdrawn in step 1155. The first outside end of the thread is placed into the outer conduit and into the channel of middle conduit in step 1160. The outer conduit is slid towards the target area, gradually moving the placed thread into the middle conduit through the channel of the middle conduit. Both ends of the looping thread will be moved into the middle conduit in step 1165. The outer conduit is slid towards the proximal point of the target, where the outer part will be moved into the middle conduit through the channel in step 1170. The target is looped with the thread when the outer conduit slides into its original position in step 1175 and the remaining part of the thread is in the middle conduit.

FIG. 12 depicts an exemplary surgical device employed in an illustrative use-case scenario. The conduit complex 105 is placed at a distal spot of the target underneath the target area 120 inside the body. The conduit complex 105 may, for example, be guided using ultrasound. The use of ultrasound or other tools that may view anatomy inside the boy such as CT or MRI and so forth, may, for example, advantageously provide mapping and/or guidance to the practitioner while the conduit complex 105 is inside the body underneath the skin 115. For example, the mapping and hydrodissection may enable the conduit complex 105 to reach the target area 120 without causing damage to the patient's skin 115 and inside anatomic structure, other than the insertion point.

In some embodiments, the target area 120 may, for example, be fascia or ligament compressing a nerve 1205. In some examples, this compression may cause pain, and other symptoms and dysfunctions, to the patient. The magnet 130 and the thread 125 are positioned around the target area 120. The magnet 130 and the thread 125 may, for example, be advantageously positioned to couple with the magnetic bar 135 locking system. For example, the coupling of the magnet 130 and the magnetic bar 135 may form the loop.

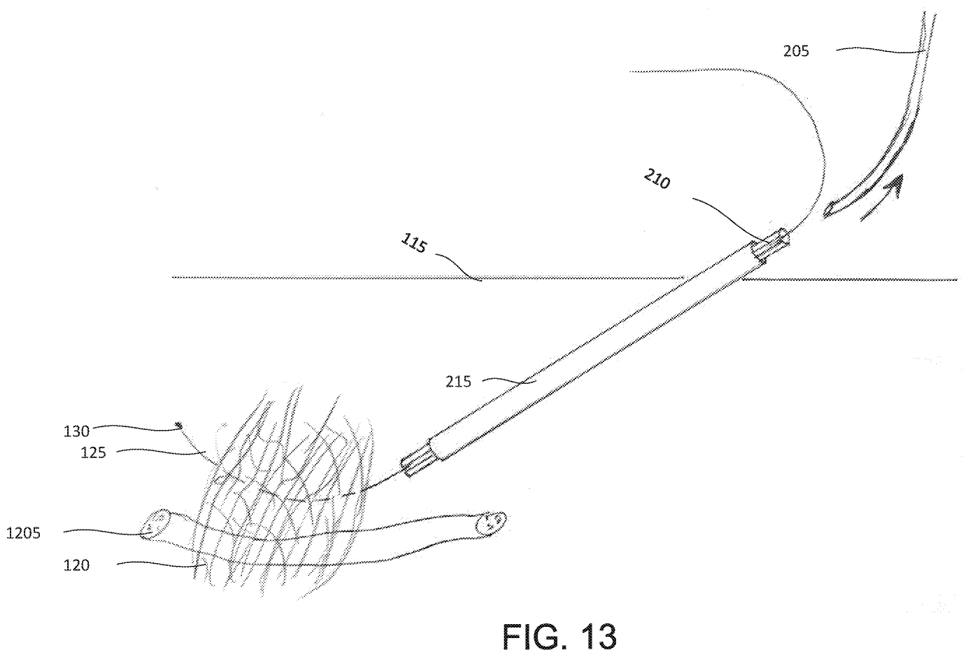

FIG. 13 depicts an exemplary surgical device employed in an illustrative use-case scenario. In some implementations, the inner conduit 205 is withdrawn from inside the body and the middle conduit 210 and the outer conduit 215 remain underneath.

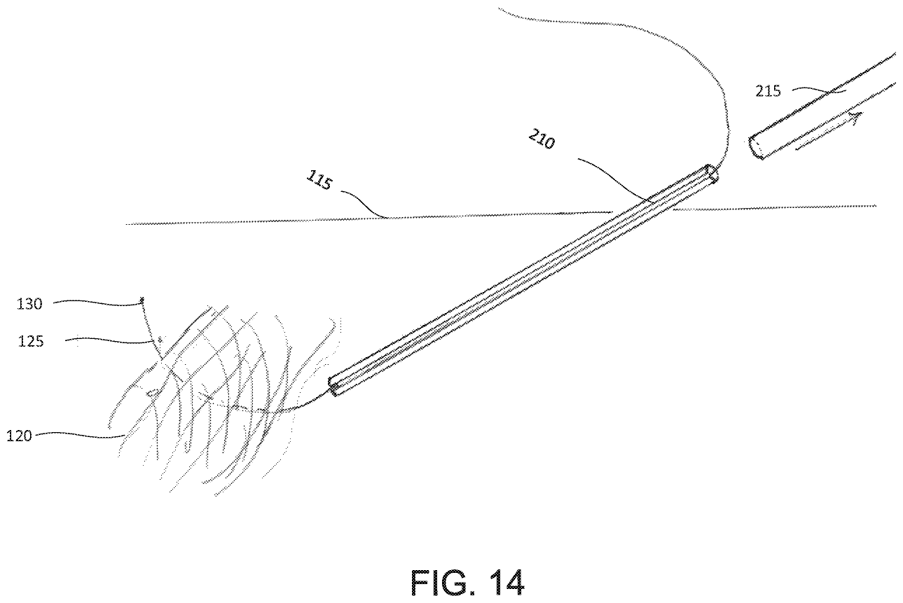

FIG. 14 depicts an exemplary surgical device employed in an illustrative use-case scenario. In various implementations, the outer conduit 215 may be removed from deep inside the body underneath the skin 115 and the middle conduit 210 may remain underneath. The thread 125 may, for example, remain in the middle conduit 210.

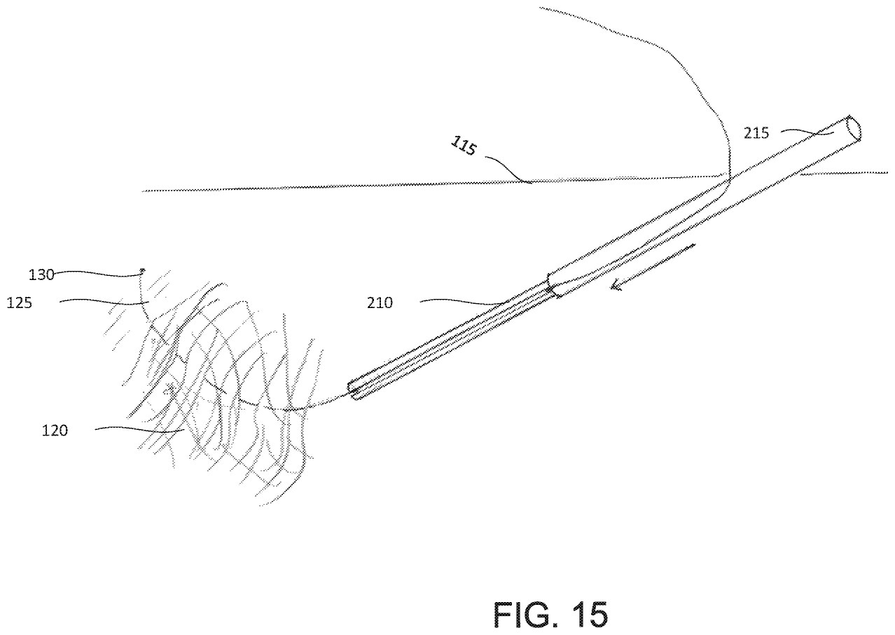

FIG. 15 depicts an exemplary surgical device employed in an illustrative use-case scenario. The outer conduit 215 is slid back on the middle conduit 210. The outer conduit 215 may, for example, be configured to move the thread 125 from the middle conduit 210 to the inside of the body of the patient while the outer conduit 215 is slid on. This may, for example, advantageously enable the conduit complex 105 to be ready for looping the upper part of the target area 120.

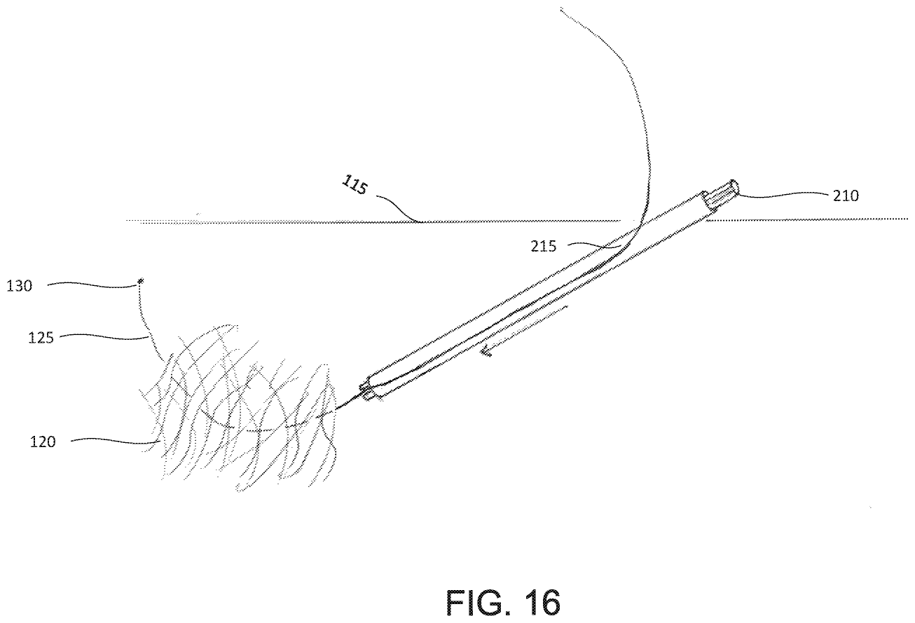

FIG. 16 depicts an exemplary surgical device employed in an illustrative use-case scenario. The thread 125 is positioned inside the patient and outside of the conduits when the outer conduit 215 returns to its original position. This may, for example, advantageously position the thread 125 underneath the target area 120 in order to form a loop and free conduit complex to be ready for looping the upper part of the target area 120.

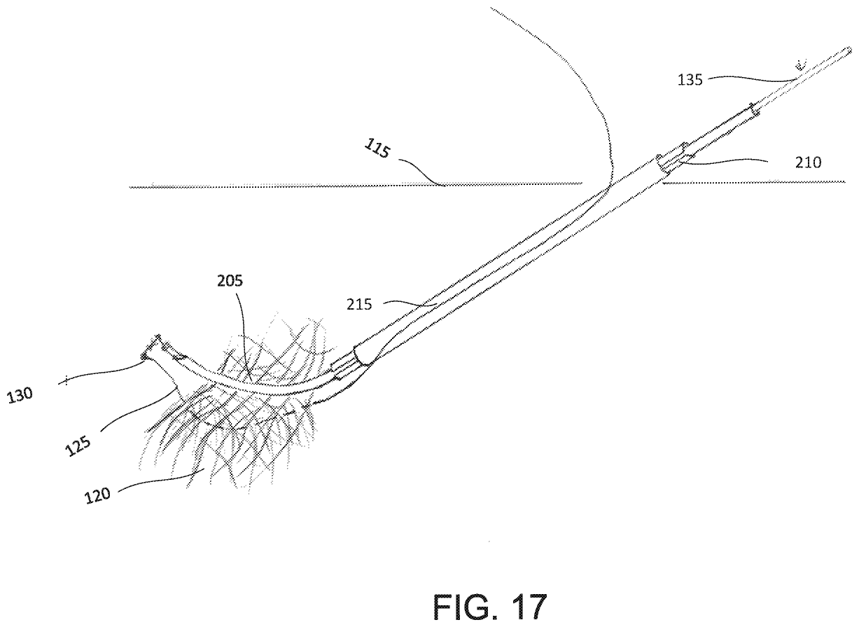

FIG. 17 depicts an exemplary surgical device employed in an illustrative use-case scenario. The conduit complex 105 is repositioned to the opposite side of the target area 120. This repositioning may, for example, advantageously allow the practitioner to couple the thread 125 with the magnetic bar 135 to complete looping of the target area 120. The magnetic bar 135 is inserted through the inner conduit 205. The magnetic bar 135 extends through the inner conduit 205 and couples with the magnet 130 and secure the thread at the tip of the magnetic bar 135 through the locking system, pulling the magnetic bar 135 outside the body and bringing the magnet 130 at the end of thread 125 to the skin surface, forming a loop around the target area 120. This loop may, for example, be used to cut the target area 120 open by reciprocal motion of two ends of the thread 125 outside the body advantageously alleviate pressure from compression on the nerve by the target area 120.

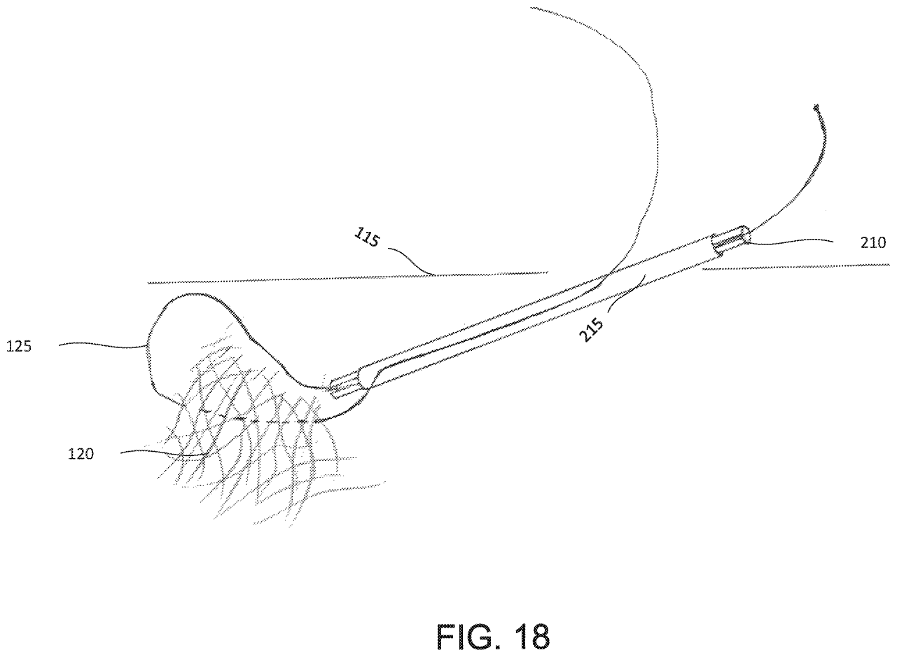

FIG. 18 depicts an exemplary surgical device employed in an illustrative use-case scenario. The magnetic bar 135 may, for example, be withdrawn from the body after forming a loop. The inner conduit 205 may, for example, be removed from the skin 115 after forming the loop. In some examples, the thread 125 may remain inside the middle conduit 210 after the magnetic bar 135 and the inner conduit 205 are removed. The thread 125 may, for example, include two ends that are above the skin.

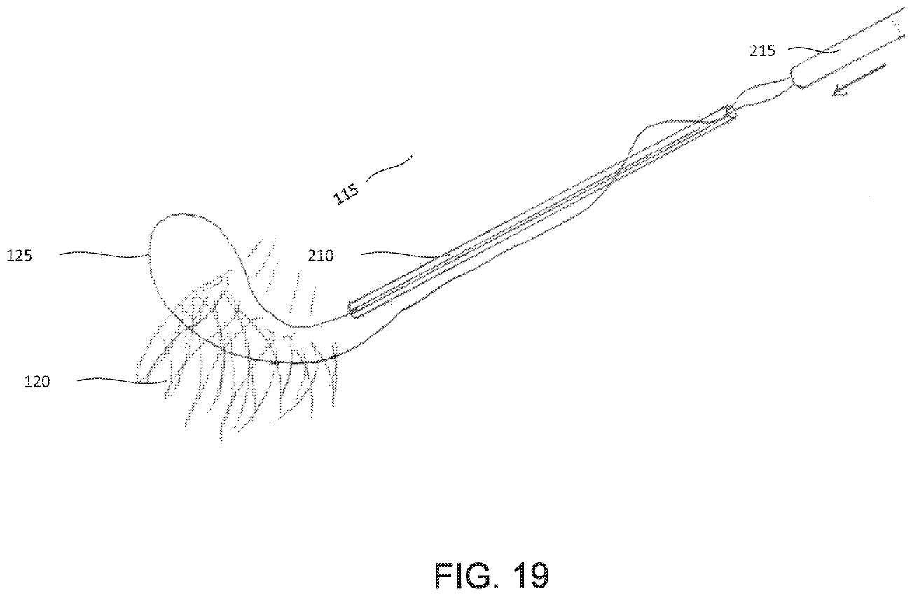

FIG. 19 depicts an exemplary surgical device employed in an illustrative use-case scenario. As depicted in FIG. 19, the thread 125 may be moved outside the middle conduit 210 to inside the middle conduit 210. The two ends of the thread 125 may, for example, reciprocally move in the middle conduit 210 when cutting the target area 120 with the thread 125. This may, for example, avoid damaging other tissue unnecessarily. In some implementations, the ends of the thread 125 may be placed into the outer conduit 215 and into the channel 505 of middle conduit 210. The outer conduit 215 may, for example, be slid towards the target area 120. For example, the sliding of the outer conduit 215 may move both ends of the thread 125 into the middle conduit 210.

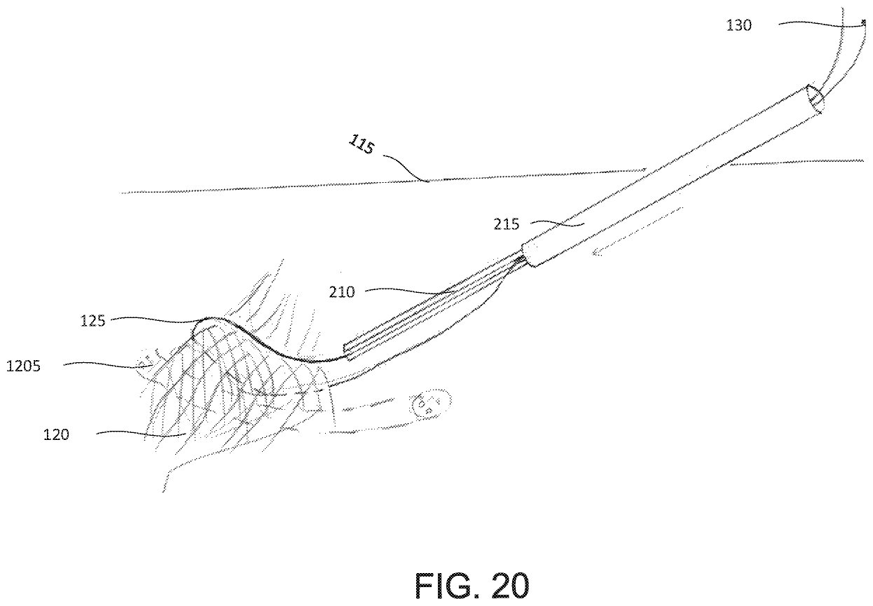

FIG. 20 depicts an exemplary surgical device employed in an illustrative use-case scenario. The outer conduit 215 may, for example, be slid towards the target area 120. This may, for example, move opposing sides of the thread into the middle conduit 210 through the channel 505.

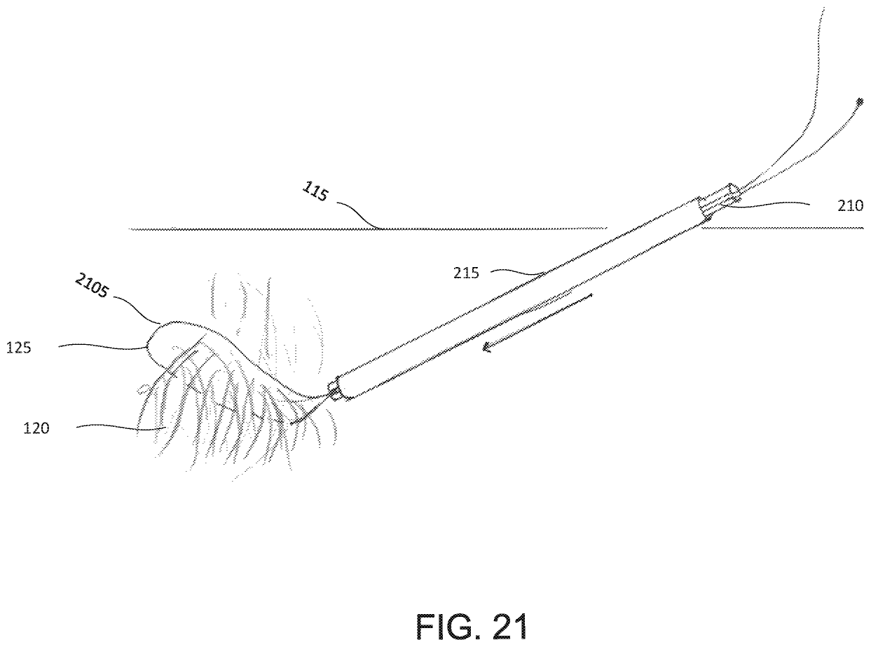

FIG. 21 depicts an exemplary surgical device employed in an illustrative use-case scenario. The outer conduit 215 may, for example, be moved to its original position. In some examples, as the outer conduit 215 is moved towards the original position, the target area 120 may be looped. The sliding of the outer conduit 215 towards its original position may, for example, form loop 2105. The loop 2105 may, for example, advantageously allow the target area 120 to be treated. In some embodiments, the target area 120 may be cut using the thread 125. This may, for example, advantageously alleviate pressure on a nerve, thus providing relief for a patient. The reciprocal cutting in the conduit may, for example, advantageously avoid causing further surrounding tissue damage. The use of the channel 505 in this process may, for example, advantageously minimize damage to the surrounding tissues.

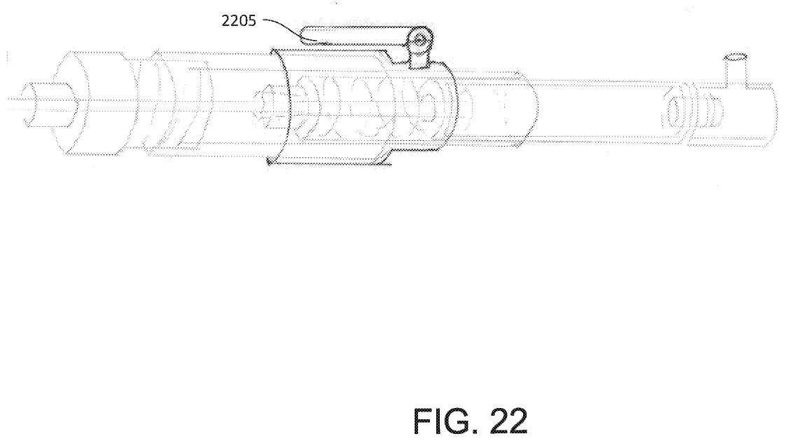

FIG. 22 depicts an exemplary securing system of a surgical device. The fluid dispenser 110 may, for example, include a securing system 2205. The securing system 2205 may, for example, be configured to immobilize the relative movement between the first shaft 220 and the second shaft 225. The securing system 2205 may be arranged between the first shaft 220 and the second shaft 225. In some embodiments, the securing system 2205 may have an open position and a closed position. The open position may, for example, be configured allow movement between the first shaft 220 and the second shaft 225. The closed position may, for example, be configured to prevent movement between the first shaft 220 and the second shaft 225. The securing system 2205 may, for example, advantageously lock the first shaft 220 and the second shaft 225. For example, this locking may prevent undesired movement of the shafts 220, 225. This may, for example, allow the practitioner to accurately control the movement of the conduit complex 105.

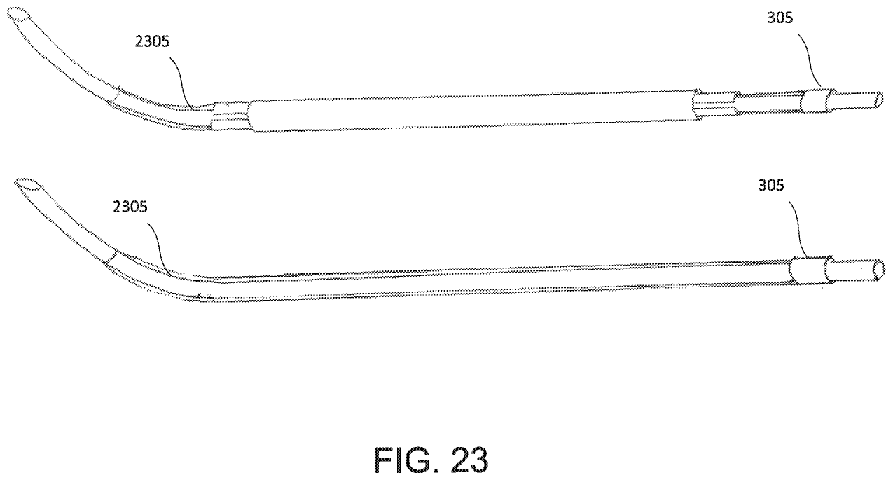

FIG. 23 depicts an exemplary tube of a surgical device. A tube 2305 is coupled to the inner conduit 205. The tube may, for example, be telescopically engaged with the inner conduit 205. The telescopic engagement of the tube 2305 and the inner conduit 205 may, for example, be tight. The tight coupling may, for example, advantageously avoid friction between the inner conduit 205 and the middle conduit 210. This friction may, for example, be avoided at the channel portion of the tip of the middle conduit 210. The tube 2305 may, for example, allow the inner conduit 205 to rotate only in the tube 2305. For example, there may be no relative motion between the inner conduit 205 and the middle conduit 210.

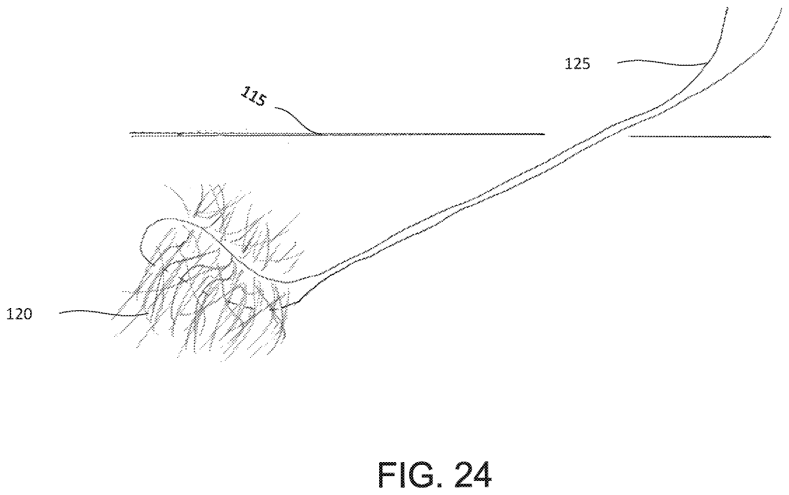

FIG. 24 depicts an exemplary surgical device employed in an illustrative use-case scenario. In some implementations, the conduit complex 105 may be used without the channel 505 defined in the middle conduit 210, as is shown in FIG. 21. This may, for example, advantageously simplify the looping process of reciprocal cutting as the thread 125 may be easily manipulated in different directions and the channel 505 is no longer required. For example, the thread 125 may not be restricted by the channel 505.

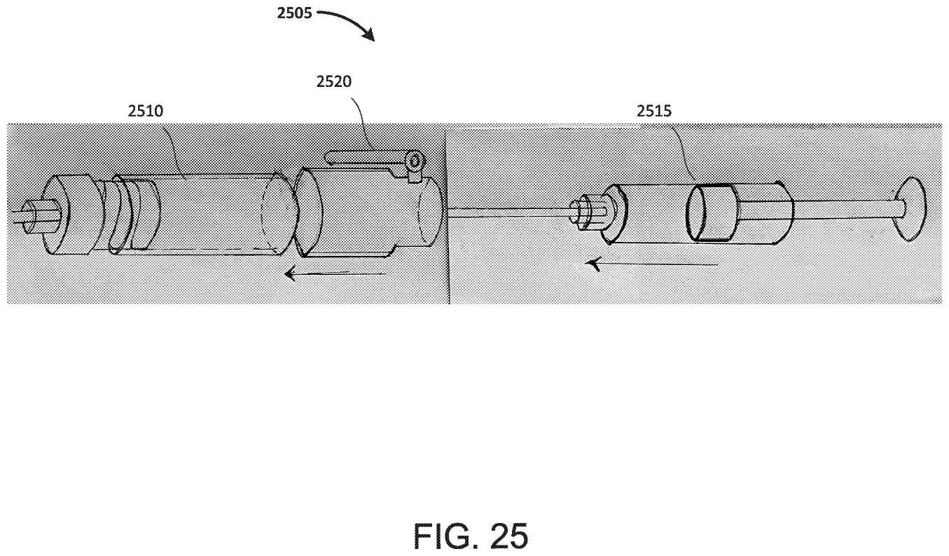

FIG. 25 depict an exemplary conduit complex. A conduit complex 2505 includes a first portion 2510 and a second portion 2515. The first portion 2510 may, for example, be similar to the first shaft 220. The second portion 2515 may, for example, be a syringe. The first portion 2510 includes a connection system 2520. The connection system 2520 may, for example, be configured to function similarly to the securing system 2205. The connection system 2520, may, for example, be configured to immobilize the relative movement between the first portion 2510 and the second portion 2515. The securing system 2205 may be arranged between the first portion 2510 and the second portion 2515. In some embodiments, the securing system 2205 may have an open position and a closed position. The open position may, for example, be configured allow movement between the first portion 2510 and the second portion 2515. The closed position may, for example, be configured to prevent movement between the first portion 2510 and the second portion 2515. The connection system 2520 may, for example, advantageously lock the first portion 2510 and the second portion 2515. For example, this locking may prevent undesired movement of the portions 2510, 2515. This may, for example, allow the practitioner to accurately control the movement of the conduit complex 2505.

In some examples, the conduit complex 2505 may be used for targeting of a deep body injection or hydrodissection. For example, the conduit complex 2505 may be used to apply medication around a nerve. The conduit complex may, for example, be unable to continue to dispense fluid in the body after fluid in the syringe runs out.

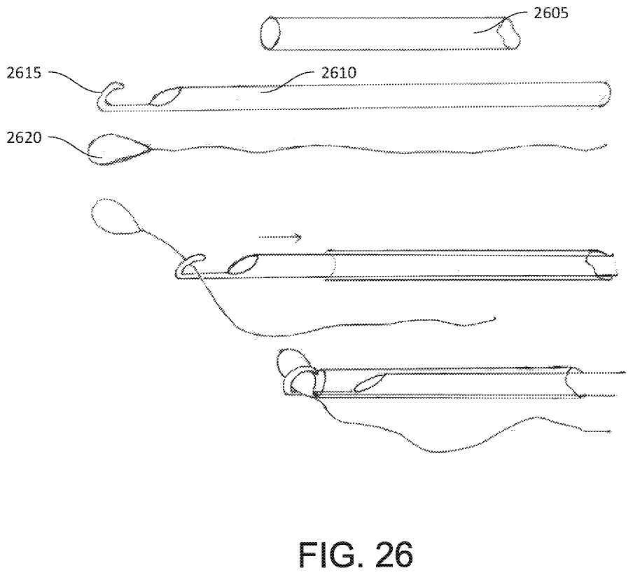

FIG. 26 depicts an illustrative hydrodissection tube with an end-hook and end-knob thread looping system. The device includes a secure tube 2605, a hydrodissection tube 2610, an end-hook 2615 formed at a distal end of the hydrodissection tube 2610, and a thread 2620 having an end-knob. The secure tube 2605 is positioned over the hydrodissection tube 2610 and is configured to slide forward relative to the hydrodissection tube in order to close an opening of the end-hook 2615. Movement of the secure tube 2605 in a forward direction encloses the end-hook 2615 and secures a thread engaged by the end-hook. The hydrodissection tube 2610 includes the end-hook 2615 at its distal portion. The hydrodissection tube may be advanced through tissue using a hydrodissection maneuver to help open soft tissue and create an open space. This open space may, for example, advantageously facilitate the manipulation of the thread to complete a looping operation. The end-hook 2615 may, for example, be configured to engage the thread 2620 under guidance, including real-time ultrasound guidance. After the thread is engaged by the end-hook, the secure tube 2605 is slid forward to close the opening of the end-hook, thereby blocking and locking the end-knob of the thread at the hook region. The thread 2620 includes an end-knob configured to be captured by the end-hook 2615. Once the end-knob is locked at the end-hook, withdrawal of the device causes the thread within the body to be pulled out, thereby finishing the target looping.

FIG. 27 depicts an illustrative surgical device for picking up an end of a thread within a body to form target looping. In some implementations, the surgical device may be referred to as a chopstick device. The device includes a first arm 2705 being formed of a thin metal or other material and includes two bending spots, 2735 and 2740, in contralateral directions. Cooperating with the first arm 2705 is a hydrodissection tube 2710, which forms a second arm of the chopstick device. Together, the first arm 2705 and the hydrodissection tube 2710 define an openable and closable space configured to receive an end of a thread.

In addition to functioning as the second arm, the hydrodissection tube 2710 is configured to perform hydrodissection of soft tissue surrounding the end of the thread to create space for the procedure. Movement of the first arm 2705 relative to the hydrodissection tube 2710 is controlled by a secure tube 2715 that is disposed in sliding relation with respect to the first arm 2705 and the second arm 2710.

In some implementations, the hydrodissection tube 2710 includes an elevated portion 2712. In some implementations, the elevated portion 2712 defines an aperture. The aperture of the elevated portion 2712 may, in some examples, be configured to immobilize the first arm to secure the thread firmly. This may, for example, advantageously additional securing of the thread.

The secure tube 2715 is actuated by a knob 2720, such that movement of the knob causes corresponding movement of the secure tube toward or away from the body. Sliding of the secure tube 2715 toward the body urges the first arm 2705 toward the hydrodissection tube 2710, thereby tightening and securing a thread positioned between the two arms.

In some embodiments, guidance and support for movement of the knob 2720 and the secure tube 2715 are provided by a grid body 2725. The grid body 2725 includes a gutter in which the knob 2720 slides and a tunnel configured to secure the secure tube 2715. The grid body 2725 further functions as a handle for manipulating the device. An assembled embodiment including the first arm 2705, hydrodissection tube 2710, secure tube 2715, knob 2720, and grid body 2725 is shown in FIG. 27.

In some examples, the grid body 2725 may function as a reinforcing frame that maintains relative alignment between the first arm 2705, the hydrodissection tube 2710, and the secure tube 2715 during actuation. In some implementations, the grid body 2725 may include one or more ribs, webs, thickened walls, or internal support features configured to resist bending, torsion, or unintended deflection when force is applied via the knob 2720. In various embodiments, the first arm 2705 may include a locally thickened region, stiffening segment, surface treatment, or laminated structure proximal to one or both bending spots 2735, 2740 to advantageously promote predictable bending behavior while maintaining sufficient rigidity for thread capture. In some examples, the secure tube 2715 may be dimensioned to closely support the first arm 2705 along a greater axial length, thereby limiting lateral play during sliding movement.

In various embodiments, the hydrodissection tube 2710 and the first arm 2705 may be coupled by a hinge, flexure, or elastic biasing element that assists return to an open configuration when the secure tube 2715 is retracted.

In various embodiments, the hydrodissection tube 2710 defines at least one fluid outlet port at a distal portion of the hydrodissection tube for directing fluid into soft tissue adjacent the end of the thread. This may, for example, advantageously allow the device to be advanced towards a target area. In some implementations, the hydrodissection tube 2710 may deliver fluid to perform hydrodissection of soft tissue.

In some implementations, the tunnel of the grid body 2725 may, for example, constrain the secure tube to linear translation relative to the hydrodisssction tube 2710. This may, for example, allow for enhanced control of the device.

In various examples, the surgical device may advantageously create space in soft tissue via the hydrodissection tube 2710 to enable access/positioning of the thread end. The surgical device may advantageously capture and lock the thread end between the first arm 2705 and hydrodissection tube 2710 by advancing the secure tube 2715 via the knob 2720. The surgical device, in some implementations, advantageously withdraw the device while retaining the thread end, thereby finishing the target looping.

In some examples, the gutter may include a detent configured to resist unintended movement of the knob 2720.

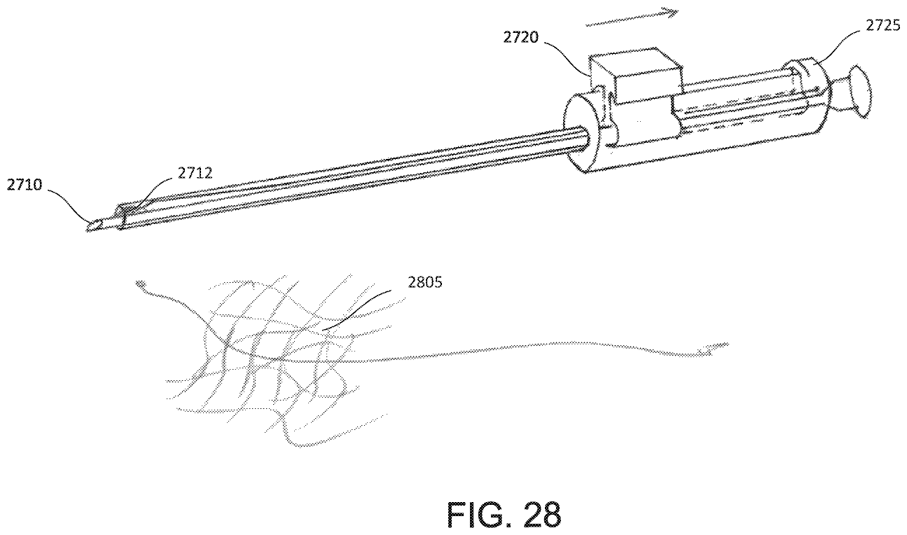

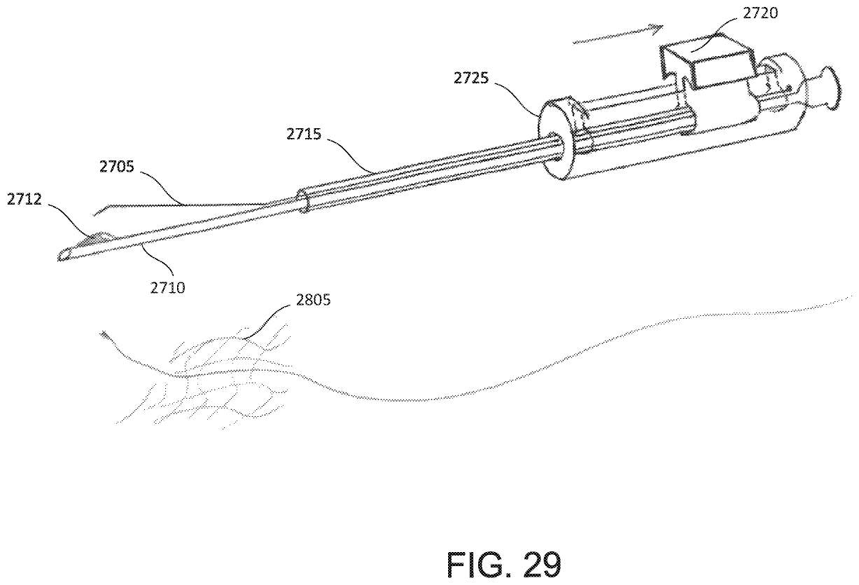

FIGS. 28-32 depict an exemplary surgical device employed in an illustrative use-case scenario. In an initial configuration (FIGS. 28 and 29), the assembled device is advanced to a target location 2805 at an end of a thread. In this configuration, the knob 2720 is positioned away from the body, which maintains separation between the first arm 2705 and the hydrodissection tube 2710 and thereby opens the chopstick configuration.

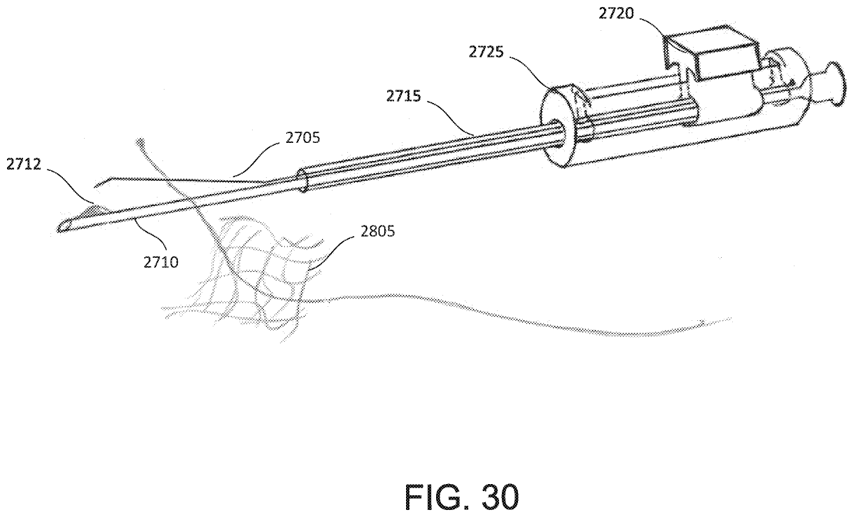

In some implementations, with the chopstick configuration opened, a hydrodissection maneuver is performed through the hydrodissection tube 2710 (FIG. 30). This maneuver hydrodissects soft tissue and enables placement of the end of the thread into the space defined between the first arm 2705 and the hydrodissection tube 2710.

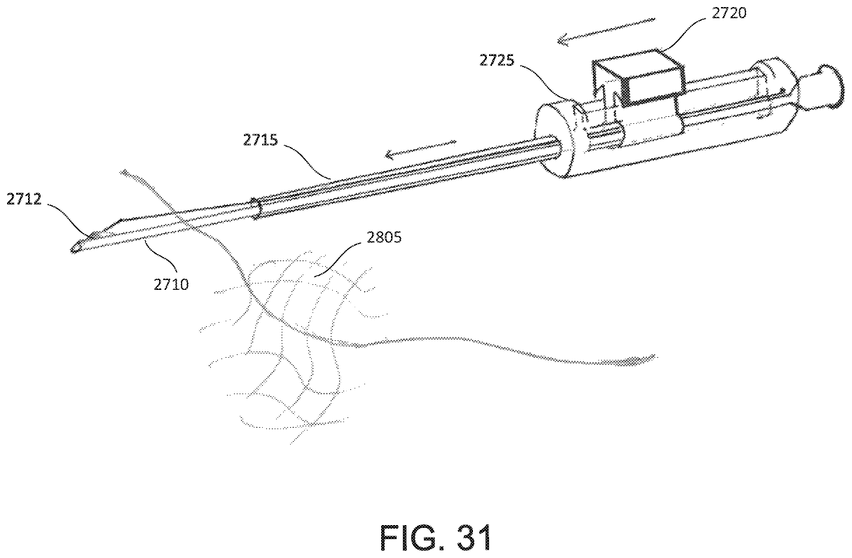

In various implementations, after the thread is positioned between the two arms, the knob 2720 may be moved toward the body (FIG. 31). Movement of the knob causes the secure tube 2715 to advance, which in turn urges the first arm 2705 toward the hydrodissection tube 2710. As the arms move closer together, the thread located between them is pushed distally. Continued advancement of the secure tube 2715 (FIG. 31) pushes the thread toward a second bending spot of the first arm 2705. Further forward movement of the secure tube forces a tip portion of the first arm to bend downward, causing the tip of the first arm 2705 to connect to a body portion of the hydrodissection tube 2710, thereby securing the thread at the bending curve of the first arm.

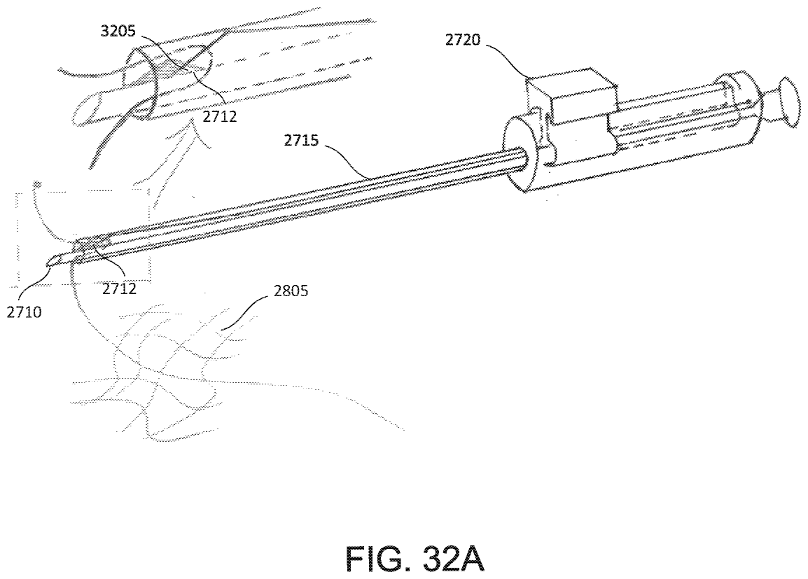

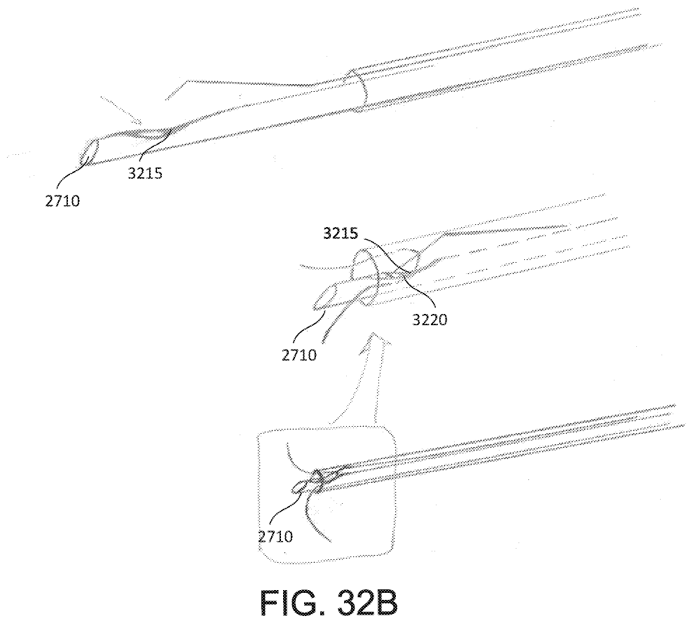

In some examples, with further movement of the knob 2720 (FIG. 32A), the thread becomes locked within the bending curve of the first arm 2705 and within a distal lumen of the secure tube 2715. The assembled device is then withdrawn from the body, carrying the end of the thread out of the body to complete the target looping. In various examples, the secure tube 2715 may be moved to bending spot 2740. The bending spots 2740 may, for example, compress to dull the angle. This may, for example, bring the tip of the first arm forward. In various examples, the tip of the first arm may be inserted into the aperture of the elevated portion 2712.

In some embodiments, an aperture 3205 of the elevated portion 2712 may be configured to immobilize the first arm 2705 to secure the thread. In some implementations, the elevated portion 2712 may dull the angle of the distal end of the hydrodissection tube. In some examples, the aperture 3205 may advantageously provide additional securing coupling to the thread to allow removal of the device from the patient.

In some embodiments, the distal portion of hydrodissection tube 2710 has a smooth concave portion 3215 (FIG. 32B). In some implementations, the smooth concave portion 3215 includes an aperture 3220 on the distal side of the concave portion. In various embodiments, the aperture 3220 of the distal side of the concave portion may be configured to immobilize the first arm 2705 to secure the thread. In some examples, the aperture 3220 may advantageously provide additional securing coupling to the thread to allow removal of the device from the patient. In various examples, the tip of the first arm 2705 may be pushed into the aperture 3220 to firmly secure the thread between the first arm 2705 and the second arm 2710 when the secure tube 2715 advances to compress the bending spots 2740 to dull the angle.

FIG. 33 depicts an exploded view of an illustrative open/close controllable loop surgical device with a hydrodissection tunnel. The device includes a hydrodissection tunnel 3305 having a loop-forming element 3315 fixed to a distal portion of the hydrodissection tunnel 3305. In some examples, the loop-forming element 3315 may be formed of metal or other solid material and configured to move back and forth to create an openable and closable loop. Formed at an end of the loop-forming element 3315 is a loop tip 3310, which may be immobilized with a distal portion of the hydrodissection tube. This may, in some example, define a potential portion of the loop between the loop-forming element and the hydrodissection tube 3305 through which a thread may pass. Movement of the loop-forming element 3315 relative to the hydrodissection tunnel 3305 is controlled by a gliding knob 3320, the gliding knob being received within a sliding groove 3340. Sliding the gliding knob 3320 forward causes the loop-forming element 3315 to advance and form an open loop. In cooperation with the loop-forming element 3315, a locking tube 3325 is configured to tighten the space between the loop-forming element and the hydrodissection tunnel 3305, resulting in a thread being locked between those components.

Control of the locking tube 3325 is provided by a sliding knob 3330, which is also received within the sliding groove 3340. The sliding groove 3340 is formed in a grip 3335, the grip connecting the hydrodissection tunnel 3305, the gliding knob 3320, and the sliding knob 3330 to allow coordinated, one-handed operation of the device.

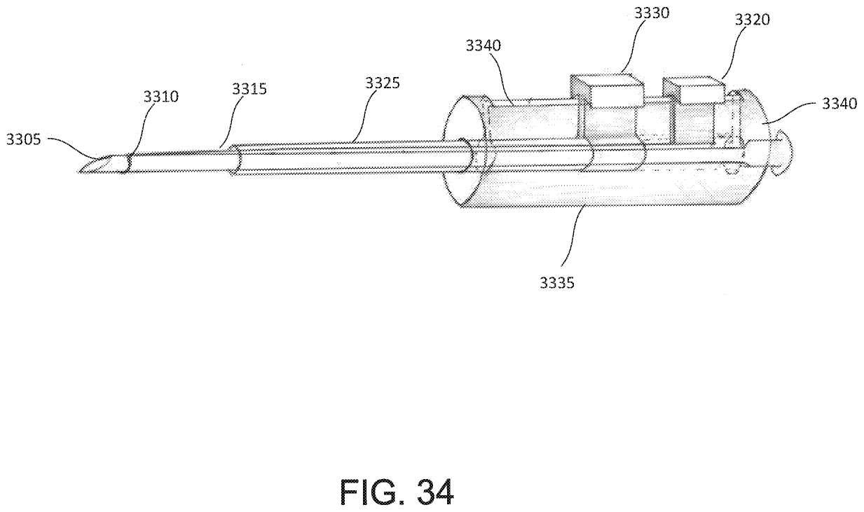

FIG. 34 depicts an assembled view of an illustrative open/close controllable loop surgical device with a hydrodissection tunnel. The gliding knob 3320 and the sliding knob 3330 are shown mounted on the grip and aligned with the sliding groove 3340, enabling coordinated control of loop formation and locking.

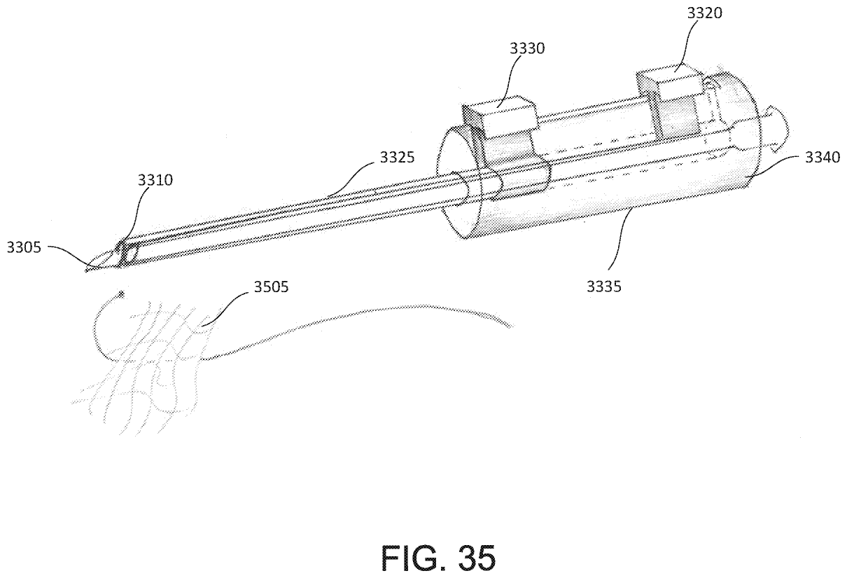

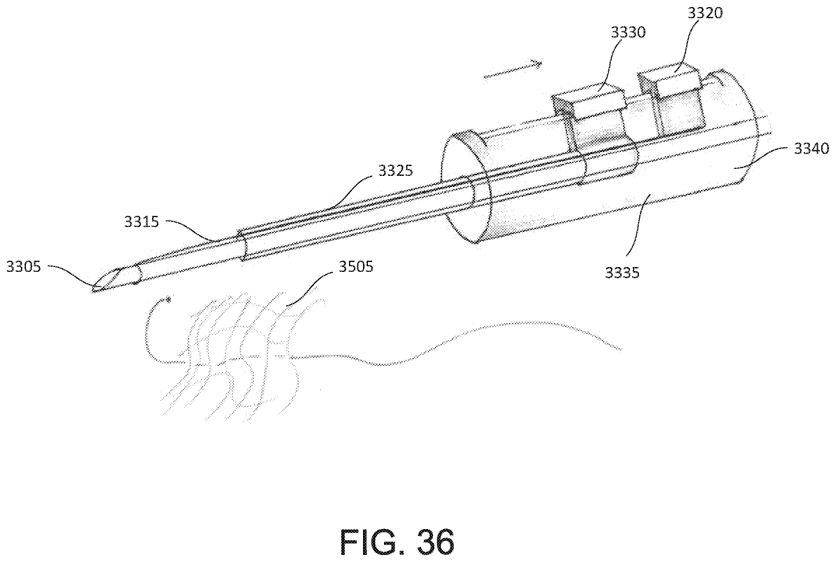

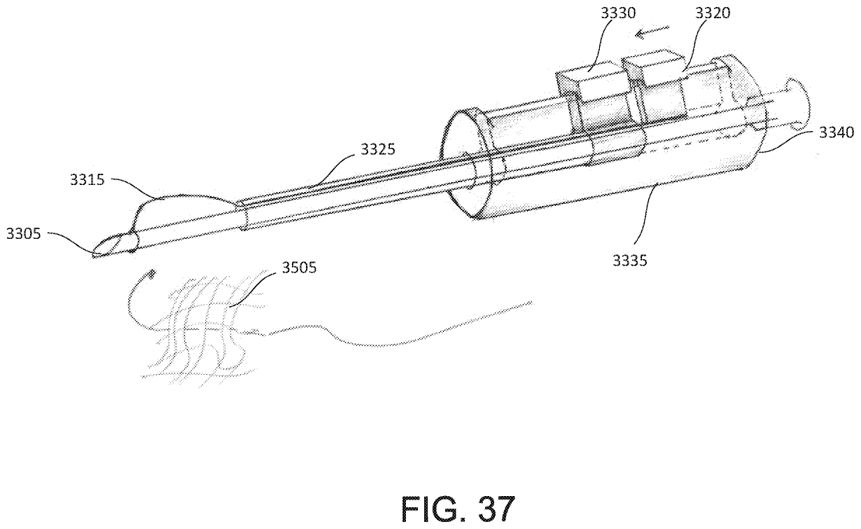

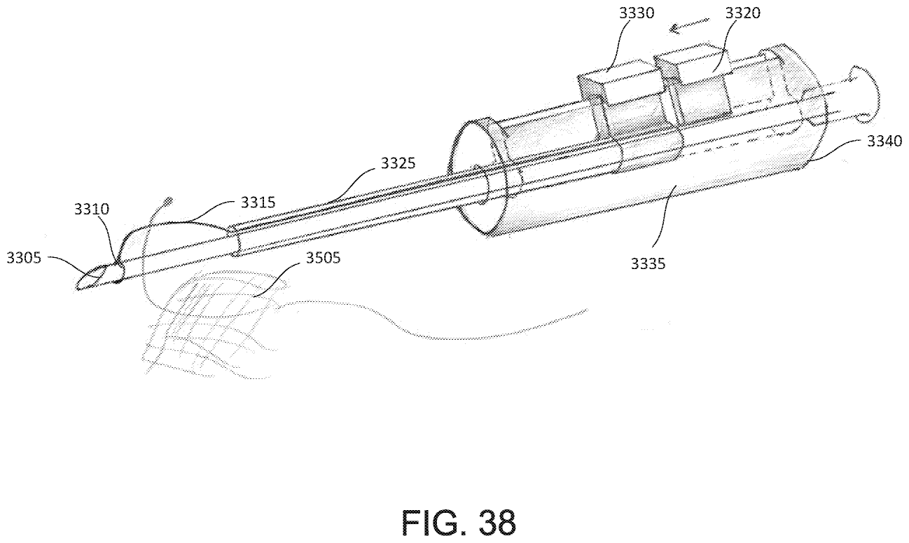

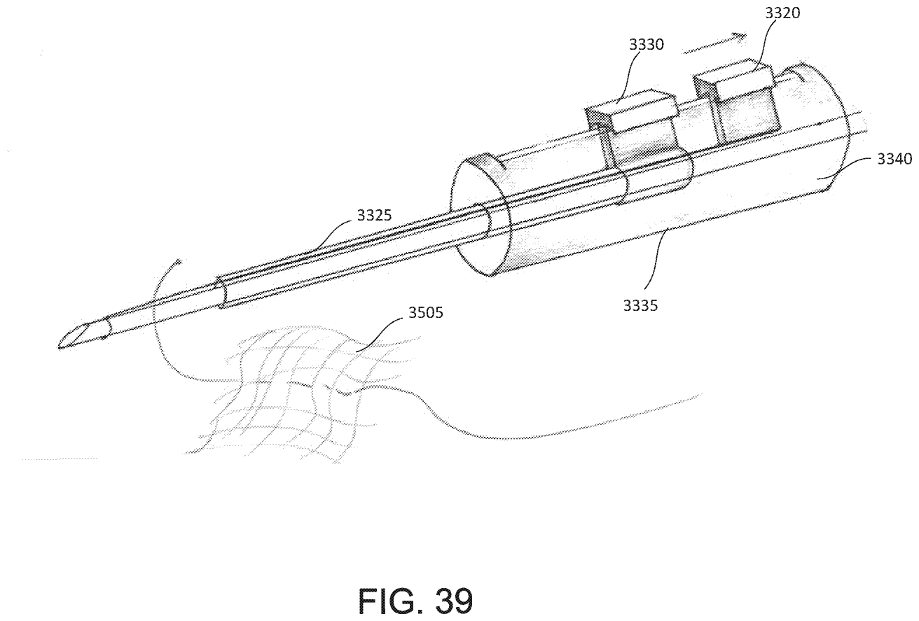

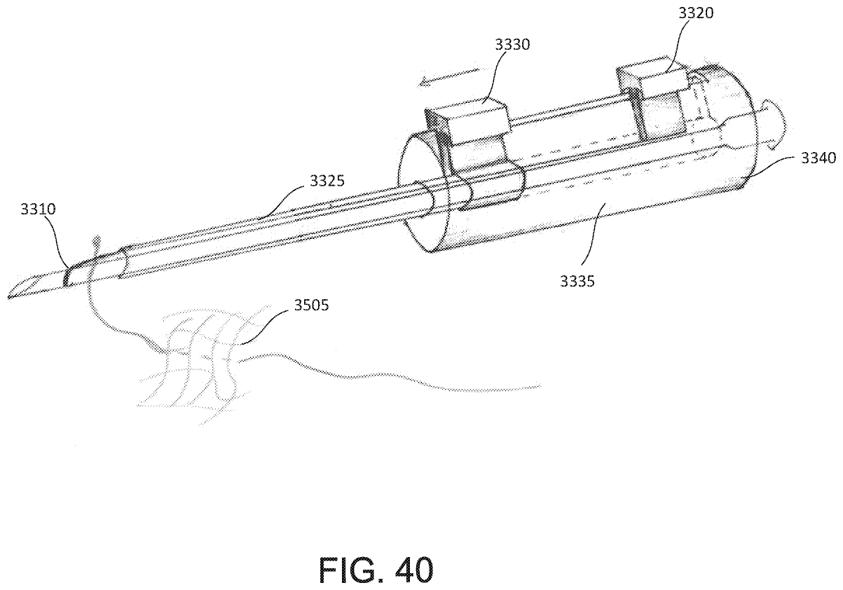

FIGS. 35-40 depict an exemplary surgical device employed in an illustrative use-case scenario. In FIG. 35, a step is depicted in which the assembled device is advanced toward an inside tip of a thread through the hydrosdissection by hydrodissection tube 3305. In this configuration, the device may, for example, be positioned such that the distal portion of the hydrodissection tunnel 3305 approaches the thread to be looped. In FIG. 36, a step is depicted in which the sliding knob 3330 is moved away from the body to expose the loop-forming element 3315. This movement positions the locking tube 3325 such that the loop-forming element is free to form an open loop. In FIG. 37, a step is depicted in which the gliding knob 3320 is slid toward the body to open the loop. As the gliding knob advances, the loop-forming element 3315 moves forward, causing the loop tip 3310 to define an open loop at the distal end of the device. In FIG. 38, a step is depicted in which the tip of the thread is passed through the open loop formed by the loop-forming element 3315 and the loop tip 3310. In FIG. 39, a step is depicted in which the gliding knob 3320 is slid away from the body to close the loop and retain the thread within the loop space. In FIG. 40, a step is depicted in which the sliding knob 3330 is moved toward the body to advance the locking tube 3325. Advancement of the locking tube tightens the space between the hydrodissection tunnel 3305 and the loop-forming element 3315, thereby securing the looping thread.

In some examples, the sequence of steps illustrated in FIGS. 35-40 advantageously provides a controlled, stepwise approach for engaging and securing a thread using simple mechanical movements while maintaining a compact device profile. In some examples, the steps allow the device to be positioned accurately at a target location 3505, selectively deploy an openable loop only when needed, capture the thread without piercing or clamping, and subsequently secure the thread through a dedicated locking action. This separation of positioning, loop opening, thread capture, loop closing, and final locking may advantageously permit each action to be performed independently and adjusted as needed, thereby promoting precision, repeatability, and stable retention while reducing the likelihood of unintended interaction or premature securing during earlier stages of the procedure.

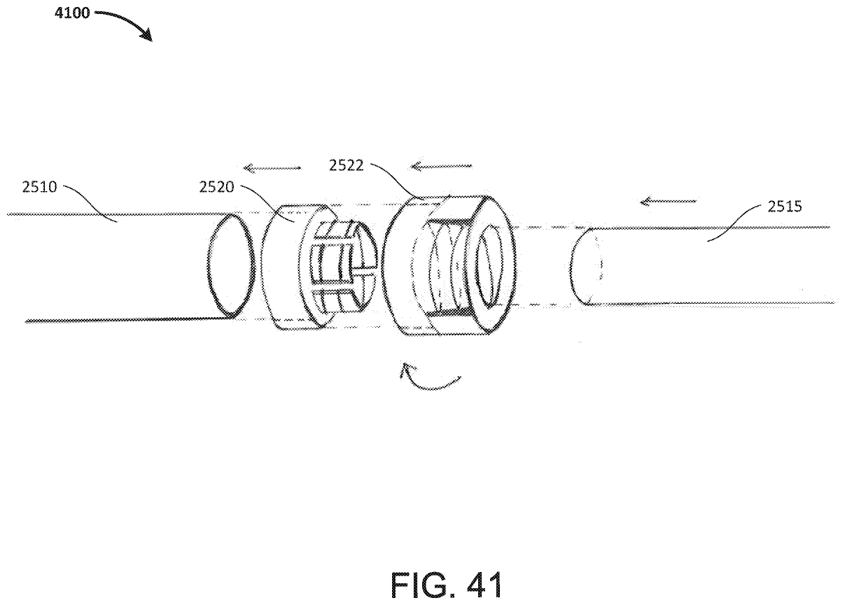

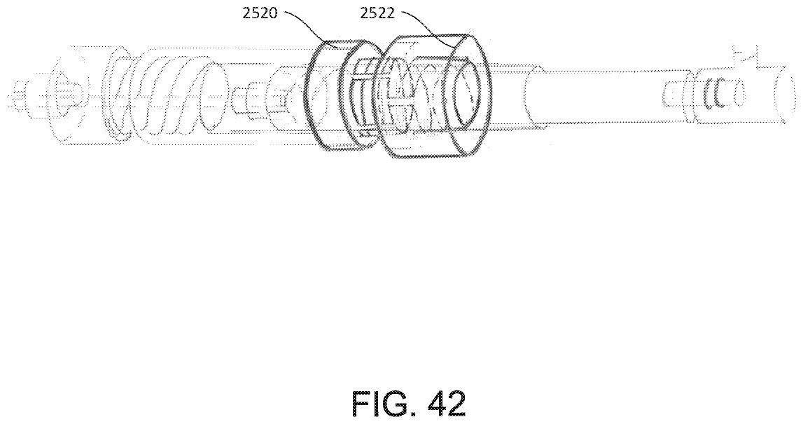

FIGS. 41 and 42 depict an exemplary attachment system of an illustrative surgical device. The fluid dispenser 110 may, for example, include a securing system 4100. The attachment system 4100 may, for example, be configured to immobilize the relative movement between the first portion 2510 and the connection system 2520. In some examples, the first portion 2510 may be immobilized with the connection system 2520. In some implementations, three lobes may arranged on the thread of the connection system 2520. The female thread 2522 may have a trapezium shape with a distal diameter smaller than the proximal diameter. In some examples, a twisting portion 2522 may be reversibly threaded onto the connection system 2520 tightly to tighten the three lobes toward the second portion 2515. This may, for example, advantageously stop relative motion between the first portion 2510 and the second portion 2515. As shown in FIG. 42, the first portion 2510 and the second portion 2515 may be assembled as such.

position may, for example, be configured to prevent movement between the first shaft 220 and the second shaft 225. The securing system 2205 may, for example, advantageously lock the first shaft 220 and the second shaft 225. For example, this locking may prevent undesired movement of the shafts 220, 225. This may, for example, allow the practitioner to accurately control the movement of the conduit complex 105.

Although various embodiments have been described with reference to the figures, other embodiments are possible.

Although an exemplary system has been described with reference to FIGS. 1-42, other implementations may be deployed in other industrial, scientific, medical, commercial, and/or residential applications.

In some embodiments, the shape of the tip of the inner conduit may be beveled. This may, for example, advantageously penetrate thick tissue such as fascia ligament.

In various embodiments, the shape of the tip of the inner conduit may be conical. This may, for example, advantageously increase the hydrodissection hydraulic pressure.

In some implementations, the shape of the tip of the inner conduit may be blunt. This may, for example, advantageously avoid nerve or vessel damage during a procedure.

In some examples, the conduit complex may be of varying sizes. Varying sizes may, for example, advantageously provide the practitioner flexibility in determining the most effective conduit complex for a particular operation. For example, an operation performed on the arm of a 5 feet tall, 130-pound patient may use a small conduit complex. In some examples, an operation performed on the leg of a 6 feet tall, 250-pound patient may use a large conduit complex.

In some embodiments, the conduit complex may be made of metal. The conduit complex may, for example, be made from stainless steel. The stainless-steel construction may, for example, advantageously provide corrosion resistance.

In some implementations, the conduit complex may be made of a shape-memory alloy. The shape-memory alloy may, for example, advantageously enable the conduit complex to return to its pre-deformed shape after it has been deformed during a procedure.

In various implementations, the conduit complex may be made of plastic. The plastic construction may, for example, advantageously be inexpensive to manufacture.

In various embodiments, the fluid dispenser and the conduit complex may be configured to operably couple. The fluid dispenser and the conduit complex may, for example, be fluidly coupled. In some examples, the shafts of the fluid dispenser may move telescopically with respect to one another.

In some embodiments, the surgical device may be guided by using ultrasound. Ultrasound may be used, for example, to create real-time images of the inside of the patient. This may, for example, advantageously provide a practitioner images of inside the patient to determine where to move the conduit complex in the patient. For example, the ultrasound may precisely and dynamically locate the target area where a loop is to be formed. The practitioner may, for example, use this guidance from the ultrasound to insert the conduit complex to initiate forming a loop with a thread.

In some implementations, the thread may be configured to cut the target area after the loop is formed. This cutting may, for example, advantageously alleviate compression on a nerve.

In various implementations, a surgical device includes a conduit complex having at least one conduit and a fluid dispenser having at least one shaft, the at least one shaft is operably coupled to the at least one conduit to dispose a thread.

In some embodiments, the middle conduit may form an entire circular perimeter. In these embodiments, the middle conduit may not define a channel.

In some examples, the device includes an adjustable loop formed at a distal portion of the wire, wherein the loop may be introduced into the body in a closed or reduced profile and subsequently expanded, contracted, or otherwise adjusted once positioned at a desired location. This capability allows the practitioner to selectively engage, capture, or manipulate tissue, sutures, or other structures with a high degree of precision. The adjustable nature of the loop enables controlled interaction with target elements while maintaining a compact insertion profile during delivery.

In some embodiments, the device may be configured to facilitate minimally invasive procedures through the use of a fine-gauge introducer and an extremely small-diameter wire. In some examples, the system may employ a needle having a gauge on the order of 21 and a wire having a diameter on the order of hundredths of a millimeter, although other dimensions may be used. This configuration may, in some implementations, advantageously allow access to target anatomical structures with reduced tissue disruption when compared to procedures requiring larger instruments or open surgical exposure. For example, the reduced profile of the system may enable placement and manipulation while limiting the size of any required access path.

In various examples, the device may include an adjustable loop formed at a distal portion of the wire, where the loop may be introduced into the body in a closed or reduced profile and subsequently expanded, contracted, or otherwise adjusted once positioned at a desired location. This capability may, in some examples, advantageously allow a practitioner to selectively engage, capture, or manipulate tissue, sutures, or other structures with a high degree of precision. For example, the adjustable nature of the loop may enable controlled interaction with target elements while maintaining a compact insertion profile during delivery.

In some embodiments, the device may incorporate a locking feature associated with the wire, such as a knob, enlargement, or other retention element positioned along the wire. The locking feature may be configured to cooperate with the loop to inhibit unintended movement, slippage, or disengagement once the loop has been adjusted to a desired configuration. This arrangement may, in some examples, advantageously promote stable engagement during manipulation without requiring complex locking mechanisms. For example, the locking feature may assist in maintaining fixation while the practitioner applies tension or positional adjustments.

In various embodiments, the system may be configured for operation using simple mechanical movements, such as telescoping motion and manual adjustment of the wire and loop, rather than relying on electronic controls or multi-component assemblies. In some examples, this mechanical simplicity may advantageously reduce operational complexity and facilitate intuitive use by a practitioner. For example, the limited number of moving parts may simplify handling, cleaning, sterilization, or maintenance when compared to more complex devices.

In some examples, the system may be adaptable for use in a variety of clinical scenarios and anatomical contexts, including procedures involving the manipulation, release, or isolation of nerves or other soft tissues. In some examples, the system may be used in nerve release procedures associated with conditions such as diabetic peripheral neuropathy, although other applications are contemplated. This versatility may advantageously allow the device to be applied in situations where existing techniques are more invasive or less precise, while remaining compatible with different procedural approaches.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A soft tissue surgical device comprising:
a first arm comprising a thin metal element having at least two contralateral bending regions;
a hydrodissection tube cooperating with the first arm and forming a second arm;
a secure tube disposed in sliding relation with respect to the first arm;
a knob coupled to the secure tube; and,
a grid body coupled to the hydrodissection tube, the grid body comprising a gutter in which the knob slides and a tunnel configured to secure the secure tube;
wherein the first arm and the hydrodissection tube cooperatively define an openable and closable space configured to receive an end of a thread;
wherein the hydrodissection tube is configured to deliver fluid to create space for positioning the end of the thread into the openable and closable space;
wherein the knob is configured to actuate sliding movement of the secure tube toward or away from the body; and,
wherein the secure tube is configured, when advanced distally, to urge the first arm toward the hydrodissection tube to tighten and secure the thread positioned between the first arm and the hydrodissection tube.

2. The soft tissue surgical device of claim 1, wherein the thin metal element comprises stainless steel.

3. The soft tissue surgical device of claim 1, wherein the at least two contralateral bending regions comprise a proximal bend and a distal bend configured to direct a distal tip portion of the first arm toward the hydrodissection tube when the secure tube is advanced distally.

4. The soft tissue surgical device of claim 1, wherein a distal end portion of the hydrodissection tube comprises a beveled or conical tip.

5. The soft tissue surgical device of claim 1, wherein the hydrodissection tube defines at least one fluid outlet port at a distal portion of the hydrodissection tube for directing fluid into soft tissue adjacent the end of the thread.

6. The soft tissue surgical device of claim 1, wherein the secure tube defines a distal lumen configured to receive the end of the thread when the secure tube is advanced distally.

7. The soft tissue surgical device of claim 1, wherein the tunnel of the grid body constrains the secure tube to linear translation relative to the hydrodissection tube.

8. The soft tissue surgical device of claim 1, wherein the gutter comprises at least one detent configured to resist unintended movement of the knob.

9. The soft tissue surgical device of claim 1, wherein the knob is configured to slide between a proximal position corresponding to an open configuration of the openable and closable space and a distal position corresponding to a closed configuration of the openable and closable space.

10. The soft tissue surgical device of claim 1, wherein the hydrodissection tube includes an elevated portion defining an aperture.

11. A soft tissue surgical device comprising:
a first arm comprising a thin metal element having at least two contralateral bending regions;
a hydrodissection tube cooperating with the first arm and forming a second arm;
a secure tube disposed in sliding relation with respect to the first arm;
a knob coupled to the secure tube; and,
a grid body coupled to the hydrodissection tube, the grid body comprising a gutter in which the knob slides and a tunnel configured to secure the secure tube;
wherein the first arm and the hydrodissection tube cooperatively define an openable and closable space configured to receive an end of a thread;
wherein the hydrodissection tube is configured to deliver fluid to create space for positioning the end of the thread into the openable and closable space; and,
wherein the knob is configured to actuate sliding movement of the secure tube toward or away from the body; and,

12. The soft tissue surgical device of claim 11, wherein the secure tube is configured, when advanced distally, to urge the first arm toward the hydrodissection tube to tighten and secure the thread positioned between the first arm and the hydrodissection tube.

13. The soft tissue surgical device of claim 11, wherein the at least two contralateral bending regions comprise a proximal bend and a distal bend configured to direct a distal tip portion of the first arm toward the hydrodissection tube when the secure tube is advanced distally.
